SEQUENCE LISTING (2 sequences)

SEQ ID NO: 1 - protein, 218 aa, Homo sapiens
CADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNP

SEQ ID NO: 2 - protein, 218 aa, Artificial Sequence
  R419N IgE-Fc
CADSNPRGVSAYLSRPSPFDLFIRKSPTITCLVVDLAPSKGTVNLTWSRASGKPVNHSTRKEEKQRNGTLTVTSTLPVGTRDWIEGETYQCNVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNP

(19) United States
(12) Patent Application Publication
(10) Pub. No.: US 2018/0273626 A1
(43) Pub. Date: Sep. 27, 2018

(54) OMALIZUMAB RESISTANT IGE VARIANTS AND THEIR USE IN ANTI-IGE THERAPY

(71) Applicant: The Board of Trustees of the Leland Stanford Junio University, Stanford, CA (US)

(72) Inventors: Theodore S. Jardetzky, San Jose, CA (US); Luke Pennington, Stanford, CA (US)

(21) Appl. No.: 15/759,188

(22) PCT Filed: Sep. 8, 2016
(86) PCT No.: PCT/US16/50831

§ 371 Date: Mar. 9, 2018

Related U.S. Application Data
(60) Provisional application No. 62/217,709, filed on Sep. 11, 2015.
(60) Provisional application No. 62/217,709, filed on Sep. 11, 2015.

(51) Int. Cl. C07K 16/28; A61K 39/395; C07K 16/42; A61K 9/00; A61K 45/06

(52) U.S. Cl. CPC C07K 16/2851; C07K 16/283; A61K 39/39566; C07K 16/4291; A61K 2039/545; A61K 45/06; A61K 39/3955; C07K 2317/41; A61K 9/0019

(57) ABSTRACT

Omalizumab-resistant IgE variants and methods of using them in combination with omalizumab for treatment of IgE-mediated disorders, including allergic diseases, inflammation, and asthma are disclosed. In particular, the invention relates to omalizumab-resistant IgE variants comprising mutations that interfere with omalizumab binding. These IgE variants can be used in combination therapy with omalizumab to effectively exchange the IgE repertoire on basophils by allowing the replacement of harmful allergic IgE species, depleted by omalizumab, with benign IgE species.

### STATEMENT REGARDING FEDERALLY SPONSORED RESEARCH OR DEVELOPMENT

This invention was made with Government support under contract W81XWH-14-1-0460 awarded by the Department of Defense and under contract AI038972 awarded by the National Institutes of Health. The Government has certain rights in the invention.

### TECHNICAL FIELD

The present invention pertains generally to anti-immunoglobulin E (IgE) therapy. In particular, the invention relates to omalizumab-resistant IgE variants and methods of using them in combination therapy with omalizumab for treatment of IgE-mediated disorders, including allergic diseases, inflammation, and asthma.

### BACKGROUND

Allergic diseases represent an overreaction of the immune system to normally benign environmental substances, such as dust mites, pet dander, pollen, or mold, and the incidence of allergies worldwide is rising at an alarming rate (Gould et al. (2008) Nat. Rev. Immunol. 8:205-217); Okada et al. (2010) Clin. Exp. Immunol. 160:1-9). IgE antibodies are central to most allergic reactions, and bind to high affinity receptors (FcεRI) present on mast cells and basophils, sensitizing these cells to respond to allergens. FcεRI is expressed as a trimer with one α-chain and two γ-chains or as a tetramer with an additional β-chain (Galli et al. (2012) Nat. Med. 18:693-704). The FcεRI α-chain (FcεRIa) binds IgE with sub-nanomolar affinity (Blank et al. (1991) J. Biol. Chem. 266:2639-2646; Garman et al. (2000) Nature 406:259-266), and cells expressing FcεRI circulate with preloaded IgE, poised for activation. A second IgE receptor (CD23) is expressed on additional cells, including B lymphocytes, where it is thought to play a role in IgE-mediated antigen presentation and feedback regulation of IgE antibody production (Yu et al. (1994) Nature 369:753-756; Getahun et al. (2005) J. Immunol. 175:1473-1482; Cooper et al. (2012) J. Immunol. 188:3199-3207; Heyman et al. (1993) Eur. J. Immunol. 23:1739-1742).

IgE has been a target for therapeutic development because of its central role in the allergic response. The anti-IgE monoclonal antibody omalizumab is currently indicated for the treatment of moderate to severe persistent asthma and chronic idiopathic urticaria. Omalizumab has demonstrated robust clinical efficacy (Busse et al. (2001) J. Allergy Clin. Immunol. 108:184-190; Braunstahl et al. (2013) Resp. Med. 107:1141-1151; Saini et al. (2015) J. Invest. Dermatol. 135:925), and has promise for a wide range of other allergic conditions, including oral food allergen desensitization regimens (Nadeau et al. (2012) Immunol. Allergy Clin. North Am. 32:111-133). Omalizumab acts primarily by neutralizing free serum IgE and gradually reducing surface levels of IgE on FcεRI expressing cells, including mast cells and basophils (Sherr et al. (1989) J. Immunol. 142:481-489). Following the omalizumab-dependent decline in surface-bound and free IgE, cell surface levels of FcεRI also fall (Bonnefoy et al. (1995) Int. Arch. Allergy Immunol. 107:40-42; Holdom et al. (2011) Nat. Struct. Mol. Biol. 18:571-576) and blood basophils up-regulate Syk expression and show enhanced sensitivity to anti-IgE stimulation (Dhaliwal et al. (2012) Proc. Natl. Acad. Sci. USA 109:12686-12691). However, beyond these phenomena, it is not fully understood how this dramatic drop in free IgE levels perturbs homeostatic mechanisms responsible for regulating IgE production or allergic responses. Preliminary studies suggest that IgE is able to reciprocally regulate its own production through CD23 in mice and humans (Yu et al., supra; Cooper et al., supra), yet it is not clear what role this plays in humans in vivo. Furthermore, it is not clear if the decline in FcεRI expression and up-regulation of Syk in basophils is ultimately helpful, or detrimental, to omalizumab's therapeutic effect.

We recently described a novel class of IgE inhibitors, Designed Ankyrin Repeat Proteins (DARPᵢₙₛ), capable of disrupting IgE:FcεRI complexes (Wurzburg et al. (2000) Immunity 13:375-385; Busse et al. (2001) J. Allergy Clin. Immunol. 108:184-190). These agents target preformed IgE:FcεRIa complexes found on mast cells and basophils and accelerate the dissociation rate constant to release free IgE. The activated release of IgE on the surface of effector cells might prove beneficial in treating acute allergic reactions and enhance the clearance of allergen-specific IgE during anti-IgE therapy. We have demonstrated that a bivalent DARPᵢₙ (bi53_79) containing a non-competitive IgE binding domain and a disruptive competitor domain, dissociates complexes with greater efficiency in vitro and shows greater potency in blocking passive cutaneous anaphylaxis in mice bearing the human FcεRI receptor (Wurzburg et al., supra). We also observed that omalizumab was not strictly a competitive inhibitor of IgE:FcεRIa interactions, but was capable of targeting and disrupting IgE:FcεRIa complexes (Wurzburg et al., supra). The mechanism for this disruptive inhibition is incompletely understood, but we have hypothesized that the degree of structural overlap between anti-IgE inhibitors and FcεRIa in their respective complexes with IgE is a key parameter governing the efficiency of accelerating complex dissociation.

There remains a need for better methods of treating IgE-mediated allergic diseases, inflammation, and asthma.

### SUMMARY

The present invention relates to omalizumab-resistant IgE variants and methods of using them in combination therapy with omalizumab for treatment of IgE-mediated disorders, including allergic diseases, inflammation, and asthma.

In one aspect, the invention includes an omalizumab-resistant immunoglobulin E (IgE) variant comprising a heavy chain polypeptide with a substitution of an amino acid corresponding to Arg-92, numbered relative to the reference sequence of SEQ ID NO: 1, wherein the substitution interferes with binding of the IgE variant to omalizumab.

In certain embodiments, the substitution in the IgE heavy chain introduces a glycosylation site into the IgE variant. In one embodiment, the amino acid corresponding to Arg-92, numbered relative to the reference sequence of SEQ ID NO: 1, is replaced with an Asn, wherein the Asn is glycosylated.

In certain embodiments, the omalizumab-resistant IgE variant comprises the amino acid sequence of SEQ ID NO: 2 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto.

In another aspect, the invention includes a composition comprising an omalizumab-resistant immunoglobulin E (IgE) variant and a pharmaceutically acceptable excipient. In certain embodiments, the composition may further comprise one or more additional agents selected from the group consisting of an antihistamine, an antileukotriene, a corticosteroid, a bronchodilator, and an anti-IgE therapeutic agent. In one embodiment, the composition comprises omalizumab. In another embodiment, the composition comprises at least one additional anti-IgE therapeutic agent other than omalizumab.

In another aspect, the invention includes a method of performing anti-IgE therapy comprising administering to a subject a therapeutically effective amount of omalizumab in combination with a therapeutically effective amount of an omalizumab-resistant IgE variant.

The administered omalizumab-resistant IgE variant may bind to Fc receptors on the surface of blood cells, including mast cells, basophils, and IgE+, HLA-DR+, or FcεRIα lymphocytes in the subject, where the omalizumab-resistant IgE variant can exchange with or replace harmful allergy-inducing IgE.

Anti-IgE therapy may be performed with omalizumab in combination with an omalizumab-resistant IgE variant to treat IgE-mediated disorders, such as IgE-mediated allergic diseases, inflammation, and asthma. In particular, combination anti-IgE therapy with an omalizumab-resistant IgE variant may be used to treat IgE-mediated allergic reactions or allergen-induced inflammation, such as caused by any ingested or inhaled allergen, occupational allergen, environmental allergen, or any other substance that triggers a harmful IgE-mediated immune reaction.

By “therapeutically effective dose or amount” of an omalizumab-resistant IgE variant or omalizumab is intended an amount that, when the omalizumab-resistant IgE variant and omalizumab are administered in combination, brings about a positive therapeutic response with respect to treatment of an individual for an IgE-mediated disorder. By “positive therapeutic response” is intended that the individual undergoing treatment exhibits an improvement in one or more symptoms of the IgE-mediated disorder for which the individual is undergoing therapy, such as a reduction in coughing, wheezing, nasal congestion, runny nose, red eyes, hives, swelling, rash, shortness of breath, bronchial inflammation, or other IgE-mediated inflammation.

In certain embodiments, the omalizumab-resistant IgE variant is administered in multiple therapeutically effective doses. In one embodiment, the omalizumab-resistant IgE variant is administered according to a daily dosing regimen. In another embodiment, the omalizumab-resistant IgE variant is administered intermittently.

In certain embodiments, the omalizumab-resistant IgE variant is administered for a period of time before and/or after administration of the omalizumab. In one embodiment, the omalizumab-resistant IgE variant is administered for one week before the first dose of omalizumab is administered to the subject. In another embodiment, the omalizumab-resistant IgE variant is administered for one week after the last dose of omalizumab is administered to the subject.

In certain embodiments, the method further comprises treating the subject with one or more other drugs or agents for treating an IgE-mediated disorder, such as, but not limited to, an antihistamine, an antileukotriene, a corticosteroid, a bronchodilator, or an anti-IgE therapeutic agent.

Any appropriate mode of administration may be used. In one embodiment, the omalizumab-resistant IgE variant is administered subcutaneously,

In another aspect, the invention includes a kit comprising a composition comprising an omalizumab-resistant IgE variant and instructions for treating an IgE-mediated disorder. The composition in the kit may further comprise a pharmaceutically acceptable excipient. The kit may also further comprise omalizumab and optionally one or more other drugs for treating an IgE-mediated disorder order. Additionally, the kit may further comprise means for delivering the composition to a subject.

In another aspect, the invention includes an omalizumab-resistant immunoglobulin E (IgE) variant comprising a heavy chain polypeptide with a substitution of an amino acid corresponding to Arg-92, numbered relative to the reference sequence of SEQ ID NO: 1, wherein the substitution interferes with binding of the IgE variant to omalizumab, for use in the treatment of an IgE-mediated disorder.

In another aspect, the invention includes an omalizumab-resistant immunoglobulin E (IgE) variant comprising a heavy chain polypeptide with a substitution of an amino acid corresponding to Arg-92, numbered relative to the reference sequence of SEQ ID NO: 1, wherein the substitution interferes with binding of the IgE variant to omalizumab, for use in in the preparation of a medicament for the treatment of an IgE-mediated disorder.

These and other embodiments of the subject invention will readily occur to those of skill in the art in view of the disclosure herein.

### BRIEF DESCRIPTION OF THE FIGURES

FIGS. 1A-1C show the organization and conformational rearrangements of the IgE-Fc. FIG. 1A shows IgE and the relative locations of the FcεRIa- (dark gray) and CD23-binding sites (medium gray). FIG. 1B shows a representation of open and closed conformations of the IgE-Fc₃₋₄ domains (including the IgE-G335C-Fc₃₋₄ mutant locked in a closed conformation), and a representation of the reciprocal allosteric inhibition by FcεRIα (dark gray) and CD23 (medium gray). FIG. 1C shows a schematic of the bent conformation of IgE, and the relative position of the Cε2 domains.

FIGS. 2A-2E show the IgE:omalizumab complex. FIG. 2A shows a cartoon diagram of the biological unit, showing IgE-G335C-Fc₃₋₄ and two omalizumab Fabs binding symmetric sites. FIG. 2B shows a side view of the biologic unit of the IgE:omalizumab complex showing the omalizumab-Fab approaching perpendicularly relative to the Cε3-Cε4 domains. FIG. 2C shows a top view of the complex revealing the two non-overlapping and symmetric omalizumab epitopes on each Cε3 domain within the IgE. FIG. 2D shows a comparison of IgE:omalizumab, FcεRIα (1F6A) and CD23 (4EZM) complexes demonstrates that omalizumab binds between CD23 and FcεRIα-binding sites within the IgE-Cε3 domain. Alignment of the IgE:omalizumab complex with the Cε3 domain of the IgE:FcεRIα complex, in dark gray, reveals no major perturbations in the FcεRIα-binding loops. FIG. 2E shows that omalizumab and Cε2 directly compete for IgE-binding sites of the surface of IgE.

FIGS. 3A-3E show the omalizumab epitope. FIG. 3A shows a top down overview of the omalizumab:IgE complex, with interface residues colored dark gray and residues shared between omalizumab:IgE and IgE:FcεRIα complexes labelled. FIG. 3B shows the positions of previously published IgE heavy chain mutations and a new IgE-Fc₃₋₄ mutation (R419N) at the omalizumab interface. Mutant residues are color-coded by their relative binding to omalizumab (medium gray 0-14%, black 15-44% and light gray 44-75% of wild-type IgE). FIGS. 3C-3E show a detailed view of the omalizumab:IgE interface, with distances (A) between atoms predicted to participate in hydrogen bonds or salt bridges shown in black. FIG. 3C shows a hydrogen bond between IgE Q417 and omalizumab light chain Y53, and a salt bridge between IgE R419 and light chain D34 and D32. FIG. 3D shows a hydrogen bond between IgE K380 and omalizumab heavy chain S31, and an intrachain salt bridge between IgE E414 and IgE R376, two residues implicated in omalizumab:IgE binding studies. FIG. 3E shows hydrogen bonds observed between IgE residues R376, A377, S378 and omalizumab heavy chain residues H101 and Y33.

FIGS. 4A-4E show the structural basis of omalizumab FcεRIα and CD23 competition. FIG. 4A shows a structural alignment of complexes, which reveals that atomic overlap between the omalizumab light chain and FcεRIα would allow omalizumab to block IgE binding at site 2. FIG. 4B shows steric conflicts between the omalizumab-Fab heavy chain and CD23 as well as direct competition for binding sites (FIG. 4C) appear to drive omalizumab inhibition of CD23:IgE interactions. FIG. 4D shows the disruptive DARPᵢₙ inhibitor E2_79 has a similar binding mode to the omalizumab Fab, yet has less atomic overlap with FcεRIα in aligned structures of the complexes. FIG. 4E shows the majority of steric clashes between omalizumab and FcεRIα and E2_79 and FcεRIα occur with N-linked glycans (black) found on FcεRIα.

FIGS. 5A-5D show that a single IgE mutation prevents omalizumab binding. FIG. 5A shows the IgE-Fc₃₋₄ point mutant, R419N, contains a novel glycosylation consensus sequence and is expressed as a soluble monomer as assessed by gel filtration chromatography of IgE-R419N-Fc₃₋₄ and IgE-Fc₃₋₄ species. FIG. 5B shows SDS-PAGE analysis of non-reduced and reduced (‘r’) IgE-R419N-Fc₃₋₄, and IgE-Fc₃₋₄, demonstrating that the R419N mutation induces an additional glycosylation event and a mass shift of ˜2 kDa per IgE chain. PNGaseF treatment removes all N-linked glycans, and demonstrates that the mass shift in the IgE-R419N-Fc₃₋₄ protein arises from N-linked glycosylation. FIGS. 5C and 5D show SPR-binding assays with immobilized omalizumab (FIG. 5C) or FcεRIα (FIG. 5D) showing that IgE-R419N-Fc₃₋₄ is unable to bind omalizumab, but exhibits binding to FcεRIα at nM concentrations.

FIGS. 6A-6D show exchange of IgE on human basophils. FIG. 6A (left panel) shows that a 2 hour treatment with 25 μM E2_79 removes surface IgE from primary human basophils, but does not alter surface FcεRIα levels within 24 hours. FIG. 6A (middle panel) shows that E2_79-treated basophils can be reloaded with JW8-IgE and tracked by JW8's mouse-l-light chain (1-LC). FIG. 6A (right panel) shows that biotinylated WT and IgE-R419N-Fc3-4 variants bind E2_79-treated basophils. FIG. 6B shows the experimental design for the IgE exchange. In brief, IgE is removed from primary basophils using E2_79. JW8-IgE is then reloaded on basophils to generate a traceable starting IgE population. FIG. 6C shows the experimental validation showing distinct populations of basophils with JW8-IgE (Q1), JW8-IgE and biotinylated IgE-Fc₃₋₄ (Q2), biotinylated IgE-Fc₃₋₄ alone (Q4) and E2_79-treated cells without labelled IgE (Q3; displaying merged dot plots from each sample). FIG. 6D in all panels shows starting JW8-reloaded population in Q1 before treatment, and cells after treatment. The left panel shows that overnight treatment with a high-concentration of omalizumab (25 μM) is sufficient to remove the majority of JW8-IgE. The middle panel shows that overnight treatment of cells with omalizumab (25 μM) and IgE-Fc₃₋₄ (1 μg ml⁻¹ or ˜18 nM) results in depletion of JW8-IgE, but no exchange for IgE-Fc₃₋₄. The right panel shows that overnight treatment of cells with omalizumab (25 μM) and IgE-R419N-Fc₃₋₄ (1 μg ml⁻¹ or ˜18 nM) results in depletion of JW8-IgE, and IgE exchange to IgE-R419N-Fc3-4. (Representative dot plots shown. N=3 at 10 μg ml⁻¹ IgE-Fc doses and controls, and N=3 at 1 μg ml⁻¹ IgE-Fc doses).

FIGS. 7A and 7B show that IgE-R419N-Fc₃₋₄ and omalizumab act synergistically. Human basophils from three healthy volunteers were isolated, stripped of native IgE by bi53_79 DARPᵢₙ treatment and reloaded with NP-reactive JW8-IgE. These NP-reactive basophils were then cultured with or without omalizumab and IgE-R419N-Fc₃₋₄ as indicated for 3 (FIG. 7A) or 6 (FIG. 7B) days before antigen challenge. Basophil activation was assessed by the percent of CD63-positive cells. There was a statistically significant difference between groups as determined by a repeated-measures analysis of variance (F=7.4, P=0.0004 Day 3 and F=19.3, P=0.0001 Day 6), and a Tukey post hoc test was used to determine the significance of differences between groups. On day 6, the untreated basophils showed reduced activation, which likely reflects the spontaneous loss of IgE.

FIGS. 8A-8F show SPR sensorgrams and kinetic data for measured IgE:omalizumab interactions. Omalizumab was immobilized on the sensor chip. The association and dissociation phases are labeled “on-rate,” and “off-rate,” respectively. FIG. 8A shows summary kinetic data for all analytes. FIG. 8B shows sensorgrams for mammalian derived IgE-Fc₃₋₄. FIG. 8C shows sensorgrams for mammalian derived IgER419N-Fc₃₋₄. FIG. 8D shows sensorgrams for insect derived IgE-Fc₃₋₄. FIG. 8E shows sensorgrams for insect derived IgEG335C-Fc₃₋₄. FIG. 8F shows sensorgrams for hybridoma derived full-length IgE Sus11.

FIGS. 9A-9F show SPR sensorgrams and kinetic data for measured IgE:FcεRIα interactions. FcεRIα was immobilized on the sensor chip. The association and dissociation phases are labeled “on-rate,” and “off-rate,” respectively. FIG. 9A shows summary kinetic data for all analytes. FIG. 9B shows sensorgrams for mammalian derived IgE-Fc₃₋₄. FIG. 9C shows sensorgrams for mammalian derived IgE-R419NFc₃₋₄. FIG. 9D shows sensorgrams for insect derived IgE-Fc₃₋₄. FIG. 9E shows sensorgrams for insect derived IgE-G335CFc₃₋₄. FIG. 9F shows sensorgrams for hybridoma derived full-length IgE Sus11.

FIGS. 10A-10C show preparation of IgE:omalizumab complexes and a representative electron density map of the IgE:omalizumab interface. FIG. 10A shows gel filtration of complex (light gray), with an excess of IgE, and IgE alone (black) FIG. 10B shows an SDS-Page gel of purified and reduced IgE-G335C-Fc₃₋₄, omalizumab-Fab, and complex (lanes 1-3) with the non-reduced complex (lane 4). FIG. 10C shows a stereo image of the electron density from a SA composite omit 2mFo-DFc map contoured at 1l near the IgE:omalizumab interface. The IgE residue R419N is labeled with an asterisk.

FIGS. 11A and 11B show that Cε2 obscures a omalizumab binding site. FIG. 11A shows that in the IgE-Fc2-4:FcεRIα complex (2Y7Q), the IgE domain Cε2 (dark gray) and receptor FcεRIα (medium gray) obscure both omalizumab-binding sites. A schematic of two adjacent IgE-Fc2-4:FcεRIα complexes on the cell surface reveals that cell bound IgE-Fc₂₋₄ could not be cross-linked by omalizumab. FIG. 11B shows a schematic of the truncated IgE-Fc₃₋₄:FcεRIα complex revealing that an omalizumab epitope in the IgE:FcεRIα complex is exposed, allowing binding to preformed complex and potentially omalizumab mediated cross linking of adjacent IgE:FcεRIα complexes. Omalizumab is depicted with the structure of a full-length mouse IgG1 antibody (1IG7) for schematic purposes only, with the heavy chain in medium gray and the light chain in light gray.

FIG. 12 shows a kinetic analysis of omalizumab binding to the IgE-Fc₃₋₄:FcεRIα complex. Previously reported binding data (Baumann et al. (2010) Immunol. Lett. 133:78-84; Eggel et al. (2014) J. Allergy Clin. Immunol. 133:1709-1719), was subject to kinetic analysis. FcεRIα was immobilized on the sensor chip, and loaded to a baseline RU with insect derived IgE-Fc₃₋₄ to generate the IgE-Fc₃₋₄:FcεRIα complex. The complex baseline runs from −67 to 0 seconds in the sensorgram. Omalizumab association is measured from 0-200 seconds, and dissociation is measured from 200-600 seconds. The data was fit with a 1:1 binding model, and the summary data is presented in the table.

FIG. 13 shows a comparison of sequence variation across human IgE heavy chain sequence sources. During validation of the omalizumab binding site, IgE heavy chain sequence variants and numbering schemes were corrected to allow for comparison across sources. Original studies with omalizumab employed sequences from the 5^{th} edition of the Sequences of Proteins of Immunological Interest. Two variants of the IgE heavy chain constant region (ID#013520 and 013521) were aligned to the Uniprot P01854 sequence. This alignment revealed several minor regions of variation, yet alignment of regions including and surrounding the omalizumab epitope, revealed 100% homology across sources, and is displayed above. Despite correcting for minor sequence differences, and adjusting numbering schemes, 3 reported mutations from Presta et al (Lung Biology in Health and Disease. Vol. 164, eds. Fick, R. B. Jr. & Jardieu, P. M., Marcel Dekker, 2002) could not be reconciled with the source sequences. All of these mutations were reported to reside in the CD loop of IgE-Fc, and correspond perfectly with crystal data if the numbering for these residues is corrected as proposed.

FIG. 14 shows the electron density at IgE-R427 and IgE-P426. Comparison of electron density in the region surrounding R427 and P426 in 2Fmo-DFc maps contoured to 1σ. Only the R427 residue within chain J has density accounting for the R427 side chain, preventing the placement of the R427 side chain in other NCS related IgE chains.

FIG. 15 shows the overlap in the binding footprint of E2_79 and omalizumab. Comparison of the footprint of omalizumab and E2_79, as defined by the residues with atomic contacts <4 Å, reveals extensive overlap.

FIGS. 16A-16D show the gating scheme for basophils from peripheral blood. Lymphocytes from the live cell population were selected by their SSC (FIG. 16A) and FSC (FIG. 16B) attributes, and subsequently CD123+ HLA-DR-cells were identified as human basophils (FIG. 16C). The basophils (light gray) as compared to the remainder of the lymphocyte population (black) also show strong staining for FcεRIα (FIG. 16D).

FIG. 17 shows summary data for IgE exchange. At left is shown data for overnight treatment of cells with omalizumab (25 μM) and IgE-Fc₃₋₄ or IgE-R419N-Fc₃₋₄ (10 μg/ml or ˜180 nM), which results in depletion of JW8-IgE, minimal exchange of IgE-Fc₃₋₄, and significant exchange for IgE-R419N-Fc₃₋₄ as assessed by the ratio of median fluorescent intensity (MFI) of treated cells to IgE-Fc₃₋₄ loading controls within the same subject (P=0.0114 paired two-tailed T-test). In the middle is shown data for an identical analysis expressing the ratio of MFIs in cells treated with omalizumab (25 μM) and IgE-Fc₃₋₄ or IgE-R419N-Fc₃₋₄ (1 μg/ml or ˜18 nM), which reveals that virtually no repertoire exchange occurred in IgE-Fc₃₋₄ treated samples, while significant repertoire exchanged occurred in samples treated with IgE-R419N-Fc₃₋₄ (P=0.0119 paired two tailed T-test). At right, it is shown that this effect is more pronounced in the low dose samples when comparing populations of IgE positive cells, gating biotin-IgE-Fc untreated cells as the IgE negative population.

### DETAILED DESCRIPTION

The practice of the present invention will employ, unless otherwise indicated, conventional methods of medicine, chemistry, and biochemistry within the skill of the art. Such techniques are explained fully in the literature. See, e.g., Ige and Anti-Ige Therapy in Asthma and Allergic Disease (Lung Biology in Health and Disease, R. B. Fick and P. M. Jardieu eds., CRC Press, 2002); Middleton's Allergy: Principles and Practice (N. F. Adkinson, B. S. Bochner, A. W. Burks, W. W. Busse, S. T. Holgate, R. F. Lemanske, and R. E. O'Hehir eds., Saunders, 8^{th} edition, 2013); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “an antibody” includes two or more antibodies, and the like.

The term “about,” particularly in reference to a given quantity, is meant to encompass deviations of plus or minus five percent.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

An IgE polynucleotide, nucleic acid, oligonucleotide, protein, polypeptide, or peptide refers to a molecule derived from any source. The molecule need not be physically derived from an organism, but may be synthetically or recombinantly produced. A number of IgE nucleic acid and protein sequences are known. A representative sequence of the constant domain of an IgE heavy chain, including residues 328 to 545 of the heavy chain, is shown in SEQ ID NO: 1 (numbering of residues according to S. C. Garman, B. A. Wurzburg, S. S. Tarchevskaya, J. P. Kinet, T. S. Jardetzky, Structure of the Fc fragment of human IgE bound to its high-affinity receptor Fc epsilonRI alpha. Nature 406, 259-266 (2000), herein incorporated by reference). In addition, a sequence of the constant domain of an IgE heavy chain comprising an Asn mutation, which confers resistance to omalizumab, is shown in SEQ ID NO: 2. Additional representative IgE sequences are listed in the National Center for Biotechnology Information (NCBI) database. See, for example, NCBI entries: Accession Nos. P01854, P01855, and P06336; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct an omalizumab-resistant IgE variant, as described herein.

The term “derived from” is used herein to identify the original source of a molecule but is not meant to limit the method by which the molecule is made which can be, for example, by chemical synthesis or recombinant means.

The terms “variant,” “analog” and “mutein” refer to biologically active derivatives of the reference molecule that retain desired activity, such as IgE activity when used in combination with omalizumab in the treatment of IgE-mediated disorders as described herein. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem Biol. (2000) 7:463-473; and Simon et al., Proc. Natl. Acad. Sci. USA (1992) 89:9367-9371 for descriptions of peptoids). Preferably, the analog or mutein has at least the same IgE activity as the native molecule. Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full-length sequence and structure. The fragment can include a C-terminal deletion an N-terminal deletion, and/or an internal deletion of the native polypeptide. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains biological activity, such as IgE activity, as defined herein.

“Substantially purified” generally refers to isolation of a substance (compound, polynucleotide, protein, polypeptide, polypeptide composition) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide molecules. Two nucleic acid, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The term “antibody” or “immunoglobulin” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

“IgE-mediated disorders” include IgE-mediated allergic diseases, inflammation, and asthma, such as, but not limited to, allergic and atopic asthma, atopic dermatitis and eczema, allergic rhinitis, allergic conjunctivitis and rhinoconjunctivitis, allergic encephalomyelitis, allergic vasculitis, anaphylactic shock, allergies, such as, but not limited to, an animal allergy (e.g., cat), a cockroach allergy, a tick allergy, a dust mite allergy, an insect sting allergy (e.g. (bee, wasp, and others), a food allergy (e.g., strawberries and other fruits and vegetables, peanuts, soy, and other legumes, walnuts and other treenuts, shellfish and other seafood, milk and other dairy products, wheat and other grains, and eggs), a latex allergy, a medication allergy (e.g., penicillin, carboplatin), mold and fungi allergies (e.g., Alternaria alternata, Aspergillus and others), a pollen allergy (e.g., ragweed, Bermuda grass, Russian thistle, oak, rye, and others), and a metal allergy. The term is meant to encompass any IgE-mediated allergic reaction or allergen-induced inflammation, such as caused by any ingested or inhaled allergen, occupational allergen, environmental allergen, or any other substance that triggers a harmful IgE-mediated immune reaction.

The term “treatment” or “treating” as used herein refers to the ability to ameliorate, suppress, mitigate, or eliminate the clinical symptoms of an IgE-mediated disorder. The effect may be prophylactic in terms of completely or partially preventing IgE-mediated disorders (e.g., preventing or reducing the severity of an allergic reaction or asthmatic attack when administered before exposure to an allergen) and/or may be therapeutic in terms of partially or completely suppressing IgE-mediated disorders.

By “therapeutically effective dose or amount” of an omalizumab-resistant IgE variant or omalizumab is intended an amount that, when the omalizumab-resistant IgE variant and omalizumab are administered in combination, brings about a positive therapeutic response with respect to treatment of an individual for an IgE-mediated disorder.

By “positive therapeutic response” is intended that the individual undergoing treatment exhibits an improvement in one or more symptoms of the IgE-mediated disorder for which the individual is undergoing therapy, such as a reduction in coughing, wheezing, nasal congestion, runny nose, red eyes, hives, swelling, rash, shortness of breath, bronchial inflammation, or other IgE-mediated inflammation. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a novel anti-IgE therapy. The methods utilize delivery of omalizumab in combination with an omalizumab-resistant IgE variant. Based on the crystallographic structure of the IgE:omalizumab complex, an omalizumab-resistant IgE variant was designed by introducing a point mutation into the IgE-Fc that interferes with binding of omalizumab (see Example 1). Treatment with omalizumab in combination with this omalizumab-resistant IgE variant results in exchange of the IgE repertoire on basophils as the harmful allergic IgE species, which are depleted by treatment with omalizumab, are replaced with a benign omalizumab-resistant IgE variant. Such combination treatment may be helpful in maintaining endogenous IgE-dependent regulatory mechanisms and further suppress the allergic response.

In order to further an understanding of the invention, a more detailed discussion is provided below regarding omalizumab resistant IgE variants and methods of using them in combination therapy with omalizumab for treatment of IgE-mediated allergic diseases, inflammation, and asthma.

A. Omalizumab-Resistant IgE Variants

As explained above, the methods of the present invention include administering an omalizumab-resistant IgE variant in combination with omalizumab. Omalizumab-resistant IgE variants can be designed by introducing one or more mutations into the constant region of the IgE heavy chain that interfere with binding of omalizumab.

The omalizumab-resistant IgE variants for use in the methods of the invention may be produced, for example, by recombinant techniques or synthetically, and may be derived by mutagenesis of an IgE from any source. A number of IgE nucleic acid and protein sequences are known. A representative sequence of the constant domain of an IgE heavy chain, including residues 328 to 545 of the heavy chain, is shown in SEQ ID NO: 1 (numbering of residues according to S. C. Garman, B. A. Wurzburg, S. S. Tarchevskaya, J. P. Kinet, T. S. Jardetzky, Structure of the Fc fragment of human IgE bound to its high-affinity receptor Fc epsilonRI alpha. Nature 406, 259-266 (2000), herein incorporated by reference). In addition, a sequence of the constant domain of an IgE heavy chain comprising an Asn mutation, which confers resistance to omalizumab, is shown in SEQ ID NO: 2. Additional representative IgE sequences are listed in the National Center for Biotechnology Information (NCBI) database. See, for example, NCBI entries: Accession Nos. P01854, P01855, and P06336; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct an omalizumab-resistant IgE variant, as described herein. Although any source of IgE can be utilized to practice the invention, preferably a benign, non-allergy-inducing IgE derived from a human source is used, particularly when the subject undergoing therapy is human.

The compositions useful in the methods of the invention may comprise biologically active variants of IgE, including variants of IgE from any species. Such variants should retain the desired biological activity of the native IgE. Methods are available in the art for determining whether a variant IgE retains the desired biological activity, and hence would serve as a therapeutically active component in a pharmaceutical composition. Biological activity can be measured using assays specifically designed for measuring activity of the native IgE, including assays described herein (see Example 1).

IgE variants can be prepared, for example, by introducing mutations in the cloned DNA sequence encoding the native IgE. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (2001) Molecular Cloning: A Laboratory Manual (Cold Spring Harbor Laboratory Press, 3^{rd} Edition); U.S. Pat. No. 4,873,192; and the references cited therein; herein incorporated by reference. Guidance as to appropriate amino acid substitutions that do not destroy biological activity of a peptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly⇔Ala, Val⇔Ile⇔Leu, Asp⇔Glu, Lys⇔Arg, Asn⇔Gln, and Phe⇔Trp⇔Tyr.

In constructing IgE variants, modifications are made such that variants continue to possess the desired activity. Obviously, any mutations made in the DNA encoding the IgE variant polypeptide must not place the sequence out of reading frame and preferably will not create complementary regions that could produce secondary mRNA structure.

Biologically active variants of IgE will generally have at least about 70%, preferably at least about 80%, more preferably at least about 90% to 95% or more, and most preferably at least about 98%, 99%, or more amino acid sequence identity to the amino acid sequence of a reference IgE protein, which serves as the basis for comparison. A variant may, for example, differ by as few as 1 to 15 amino acid residues, as few as 1 to 10 residues, such as 6-10, as few as 5, as few as 4, 3, 2, or even 1 amino acid residue.

With respect to optimal alignment of two amino acid sequences, the contiguous segment of the variant amino acid sequence may have the same number of amino acids, additional amino acid residues or deleted amino acid residues with respect to the reference amino acid sequence. The contiguous segment used for comparison to the reference amino acid sequence will typically include at least 8 contiguous amino acid residues, and may be 10, 12, 13, 17, 36, 40, 50, 60, 70, or more amino acid residues. Corrections for sequence identity associated with conservative residue substitutions or gaps can be made (see, e.g., Smith-Waterman homology search algorithm). A biologically active variant of an IgE polypeptide of interest may differ from the native polypeptide by as few as 1-20 amino acids, including as few as 1-15, as few as 1-10, such as 6-10, or as few as 5, including as few as 4, 3, 2, or even 1 amino acid residue.

The precise chemical structure of a protein having IgE activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of proteins having IgE activity as used herein. Further, the primary amino acid sequence of the IgE may be augmented by derivatization using sugar moieties (glycosylation), polyethylene glycol (PEG), or by other supplementary molecules such as lipids, phosphate, acetyl, methyl, or pyroglutamyl groups, and the like. It may also be augmented by conjugation with saccharides. Certain aspects of such augmentation are accomplished through post-translational processing systems of the producing host; other such modifications may be introduced in vitro. In any event, such modifications are included in the definition of an IgE polypeptide used herein as long as the IgE activity of the peptide is not destroyed. It is expected that such modifications may quantitatively or qualitatively affect the activity, either by enhancing or diminishing the activity of the IgE variant, in the various assays. Further, individual amino acid residues in the chain may be modified by oxidation, reduction, or other derivatization, and the polypeptide may be cleaved to obtain fragments that retain activity. Such alterations that do not destroy IgE activity are included in the definition of IgE variants as used herein.

In one embodiment, the omalizumab-resistant immunoglobulin E (IgE) variant comprises a heavy chain polypeptide with a substitution of an amino acid corresponding to Arg-92, numbered relative to the reference sequence of SEQ ID NO: 1, wherein the substitution interferes with binding of the IgE variant to omalizumab.

In certain embodiments, the substitution in the IgE heavy chain introduces a glycosylation site into the IgE variant. In one embodiment, the amino acid corresponding to Arg-92, numbered relative to the reference sequence of SEQ ID NO: 1, is replaced with an Asn, wherein the Asn is glycosylated.

In certain embodiments, the omalizumab-resistant IgE variant comprises the amino acid sequence of SEQ ID NO: 2 or a sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto.

B. Pharmaceutical Compositions

An omalizumab-resistant IgE variant can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the IgE variant or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the omalizumab-resistant IgE variant (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising an omalizumab-resistant IgE variant, prepared as described herein, are in unit dosage form, meaning an amount of a conjugate or composition of the invention appropriate for a single dose, in a premeasured or pre-packaged form.

The compositions herein may optionally include one or more additional agents, such as omalizumab or other drugs for treating an IgE-mediated disorder, or other medications used to treat a subject for a condition or disease. Particularly preferred are compounded preparations including an omalizumab-resistant IgE variant and omalizumab or one or more other drugs for treating an IgE-mediated disorder, such as, but not limited to, antihistamines, antileukotrienes, corticosteroids, bronchodilators, or other anti-IgE therapeutic agents. Alternatively, such agents can be contained in a separate composition from the composition comprising the omalizumab-resistant IgE variant and co-administered concurrently, before, or after the composition comprising the omalizumab-resistant IgE variant.

C. Administration

At least one therapeutically effective dose of an omalizumab-resistant IgE variant and omalizumab will be administered. By “therapeutically effective dose or amount” of an omalizumab-resistant IgE variant or omalizumab is intended an amount that, when the omalizumab-resistant IgE variant and omalizumab are administered in combination, brings about a positive therapeutic response with respect to treatment of an individual for an IgE-mediated disorder. By “positive therapeutic response” is intended the individual undergoing the combination treatment according to the invention exhibits an improvement in one or more symptoms of the IgE-mediated disorder for which the individual is undergoing therapy, such as a reduction in coughing, wheezing, nasal congestion, runny nose, red eyes, hives, swelling, rash, shortness of breath, bronchial inflammation, or other IgE-mediated inflammation.

In certain embodiments, multiple therapeutically effective doses of either the omalizumab-resistant IgE variant or omalizumab will be administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an omalizumab-resistant IgE variant and omalizumab will be administered once every two to four weeks for an extended period of time, such as for 1, 2, 3, 4, 5, 6, 7, 8 . . . 10 . . . 15 . . . 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

The omalizumab-resistant IgE variant can be administered prior to, concurrent with, or subsequent to the omalizumab. If provided at the same time as the omalizumab, the omalizumab-resistant IgE variant can be provided in the same or in a different composition. Thus, the two agents can be presented to the individual by way of concurrent therapy. By “concurrent therapy” is intended administration to a human subject such that the therapeutic effect of the combination of the substances is caused in the subject undergoing therapy. For example, concurrent therapy may be achieved by administering at least one therapeutically effective dose of a pharmaceutical composition comprising an omalizumab-resistant IgE variant and at least one therapeutically effective dose of a pharmaceutical composition comprising omalizumab according to a particular dosing regimen. Administration of the separate pharmaceutical compositions can be at the same time (i.e., simultaneously) or at different times (i.e., sequentially, in either order, on the same day, or on different days), as long as the therapeutic effect of the combination of these substances is caused in the subject undergoing therapy.

In certain embodiments, the omalizumab-resistant IgE variant is administered for a brief period prior to administration of omalizumab and continued for a brief period after treatment with omalizumab is discontinued in order to ensure that IgE levels are adequate in the subject during omalizumab therapy. For example, the omalizumab-resistant IgE variant can be administered starting one week before administration of the first dose of omalizumab and continued for one week after administration of the last dose of omalizumab to the subject.

In other embodiments of the invention, the pharmaceutical compositions comprising the agents, such as an omalizumab-resistant IgE variant and/or omalizumab are a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising the omalizumab-resistant IgE variant and omalizumab may be administered using the same or different routes of administration in accordance with any medically acceptable method known in the art. Suitable routes of administration include parenteral administration, such as subcutaneous (SC), intraperitoneal (IP), intramuscular (IM), intravenous (IV), or infusion, oral and pulmonary, nasal, topical, transdermal, and suppositories. Where the composition is administered via pulmonary delivery, the therapeutically effective dose is adjusted such that the soluble level of the agent, such as the omalizumab-resistant IgE variant or omalizumab in the bloodstream, is equivalent to that obtained with a therapeutically effective dose that is administered parenterally, for example SC, IP, IM, or IV. In some embodiments of the invention, the pharmaceutical composition comprising an omalizumab-resistant IgE variant is administered by IM or SC injection, particularly by IM or SC injection locally to the region where the omalizumab used in the anti-IgE therapy is administered. Similarly, omalizumab can be administered by IV, IM, IP or SC injection.

Factors influencing the respective amount of the various compositions to be administered include, but are not limited to, the mode of administration, the frequency of administration (i.e., daily, or intermittent administration, such as once every 2 to 4 weeks), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

Where a subject undergoing therapy in accordance with the previously mentioned dosing regimens exhibits a partial response or a relapse following a prolonged period of remission, subsequent courses of concurrent therapy may be needed to achieve complete remission of the disease. Thus, subsequent to a period of time off from a first treatment period, a subject may receive one or more additional treatment periods comprising anti-IgE therapy with omalizumab in combination with an omalizumab-resistant IgE variant. Such a period of time off between treatment periods is referred to herein as a time period of discontinuance. It is recognized that the length of the time period of discontinuance is dependent upon the degree of response (e.g., complete or partial recovery from an IgE-mediated disorder, such as an allergic disease, inflammation, or asthma) achieved with any prior treatment periods of concurrent therapy with these therapeutic agents.

D. Kits

The invention also provides kits comprising one or more containers holding compositions comprising an omalizumab-resistant IgE variant and/or omalizumab (e.g., together with the omalizumab-resistant IgE variant or separate), and optionally one or more other drugs for treating an IgE-mediated disorder. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating an IgE-mediated disorder, such as an allergic disease, inflammation, or asthma. The package insert can be an unapproved draft package insert or can be a package insert approved by the Food and Drug Administration (FDA) or other regulatory body.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Structural Basis of Omalizumab Therapy and Omalizumab-Mediated IgE Exchange

Introduction

Omalizumab received FDA approval over a decade ago; yet, no structure of the omalizumab:IgE complex has been determined. To understand the structural basis of omalizumab:IgE interactions and its ability to inhibit both FcεRI and CD23 binding, we determined the structure of the omalizumab Fab bound to a disulfide bond mutant of the IgE-Fc Cε3-Cε4 fragment (IgE-G335C-Fc₃₋₄) to 2.5 Å (Table 1). Omalizumab binds to the IgE Cε3 domains outside of the FcεRI-binding site, similar to the anti-IgE Designed Anykyrin Repeat Protein (DARPᵢₙ) E2_79, in good agreement with prior mapping studies of the epitope (Baumann et al. (2010) Immunol. Lett. 133:78-84; Kim et al. (2012) Nature 491:613-617; Presta, L. & Shields, R. in Lung Biology in Health and Disease. Vol. 164 (eds Fick, R. B. Jr. & Jardieu, P. M.) (Marcel Dekker, 2002)). The complex structure clarifies how omalizumab blocks IgE interactions with both the high- and low-affinity receptors. Despite the similarity in omalizumab and E2_79-binding sites on IgE, E2_79 is a disruptive inhibitor that can accelerate the dissociation of IgE:FcεRI complexes, while omalizumab has only poor (˜1,000-fold weaker) disruptive activity (Kim et al., supra; Eggel et al. (2014) J. Allergy Clin. Immunol. 133:1709-1719). Comparison of the omalizumab and E2_79 IgE complexes provides insights into the mechanism of disruptive inhibition, which could help in the development of anti-IgE antibodies with improved disruptive capabilities.

The omalizumab structure also facilitated the design of an IgE-Fc₃₋₄ mutant (IgE-R419N-Fc₃₋₄) that is resistant to omalizumab neutralization but is able to bind CD23 and FcεRI. Significant experimental evidence has accumulated suggesting that IgE-dependent homeostatic regulatory pathways respond to the loss of receptor-bound IgE induced by omalizumab treatment, and could offset or constrain the therapeutic benefit of the anti-IgE treatment (Cooper et al., supra; Bonnefoy et al., supra; Prussin et al. (2003) J. Allergy Clin. Immunol. 112:1147-1154; MacGlashan et al. (1997) J. Immunol. 158:1438-1445; Zaidi et al. (2010) J. Allergy Clin. Immunol. 125:902-908 e907; MacGlashan et al. (2012) J. Allergy Clin. Immunol. 130:1130-1135; Macglashan et al. (2013) J. Allergy Clin. Immunol. 132:906-911; Aubry et al. (1992) Nature 358:505-507; Fellmann et al. (2015) Immun. Inflamm. Dis. 3:339-349). We demonstrate that the IgE-R419N-Fc₃₋₄ mutant, in combination with omalizumab, can effectively exchange cell-bound IgE with IgE-R419N-Fc₃₋₄ and that this dual inhibitor treatment is more potent at blocking basophil activation than either inhibitor alone. This approach of simultaneously depleting antigen-specific IgE, while engaging FcεRI and CD23 receptors with an IgE variant, can be used to further probe the role of IgE-dependent regulatory pathways during anti-IgE treatment and may provide a route to enhance current anti-IgE therapies.

Results

Structure of the IgE-Omalizumab Complex.

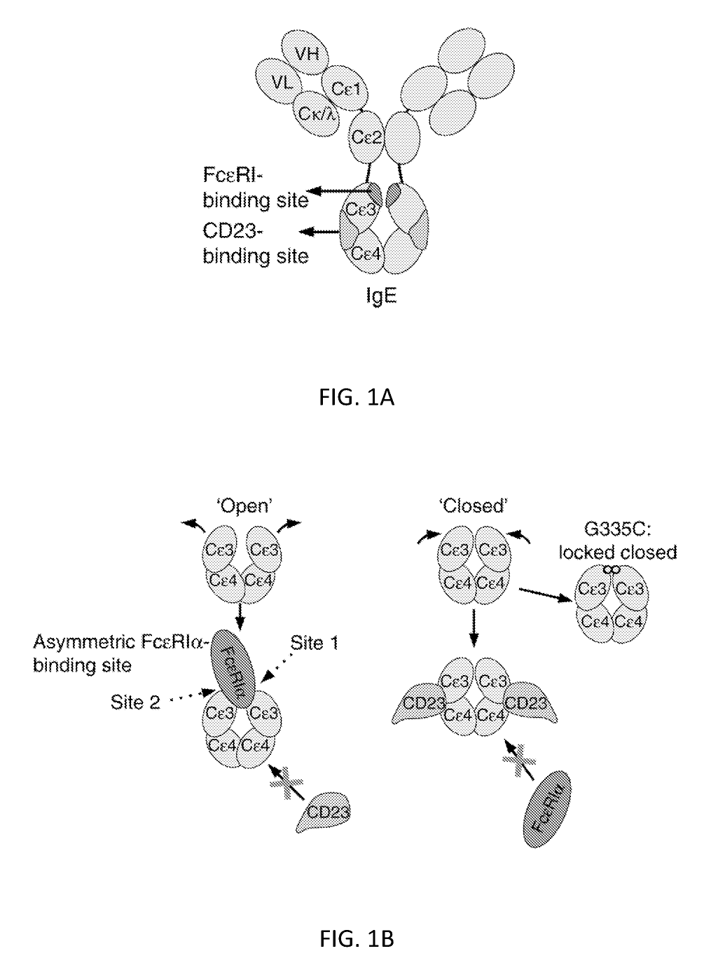

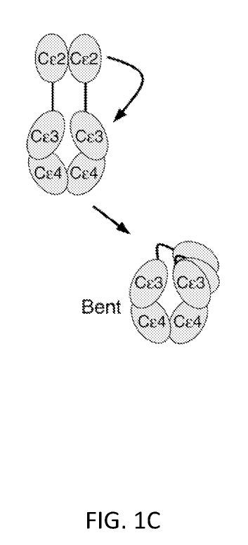

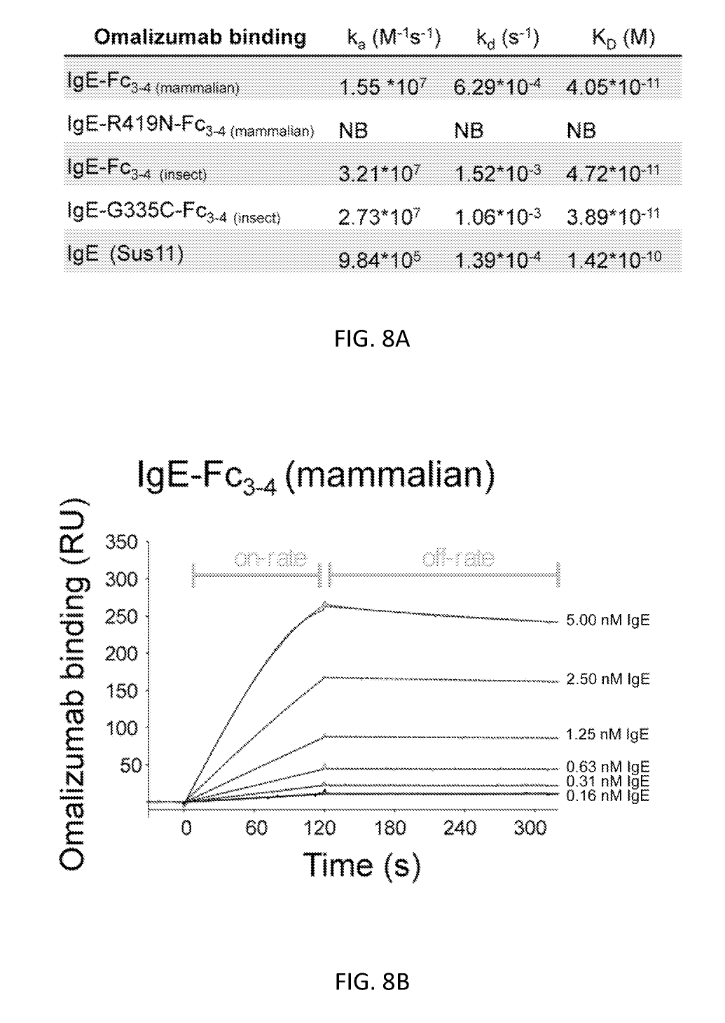

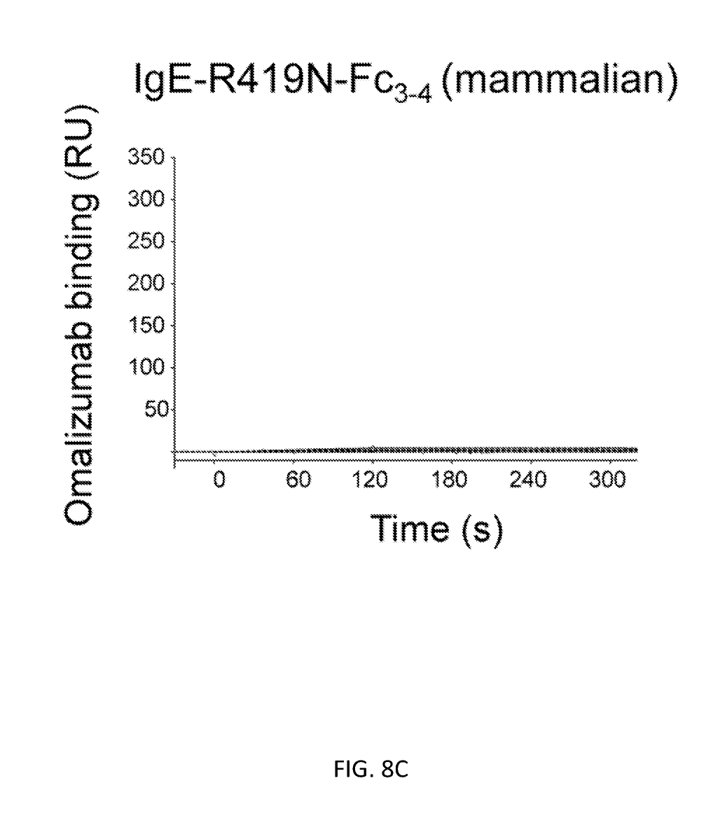

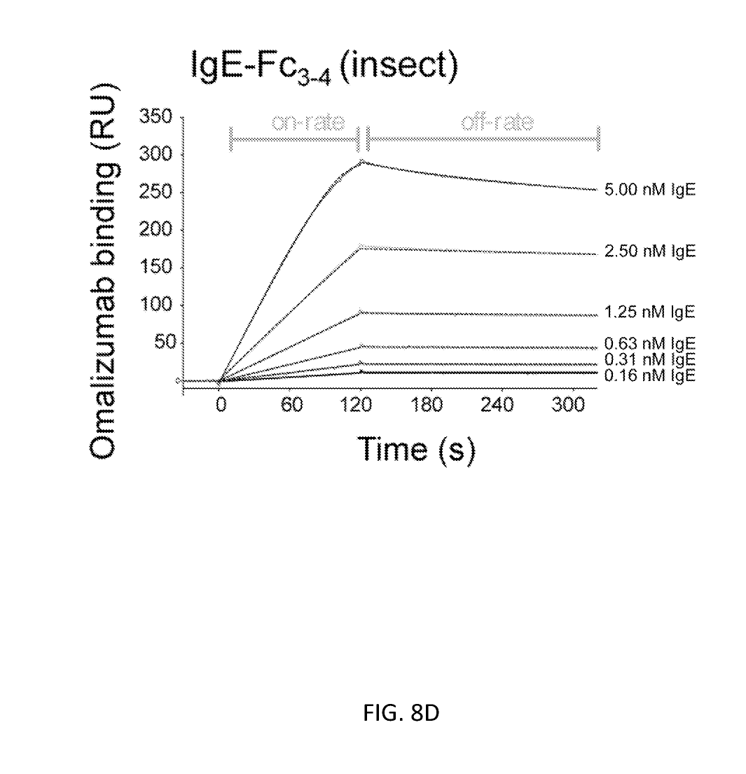

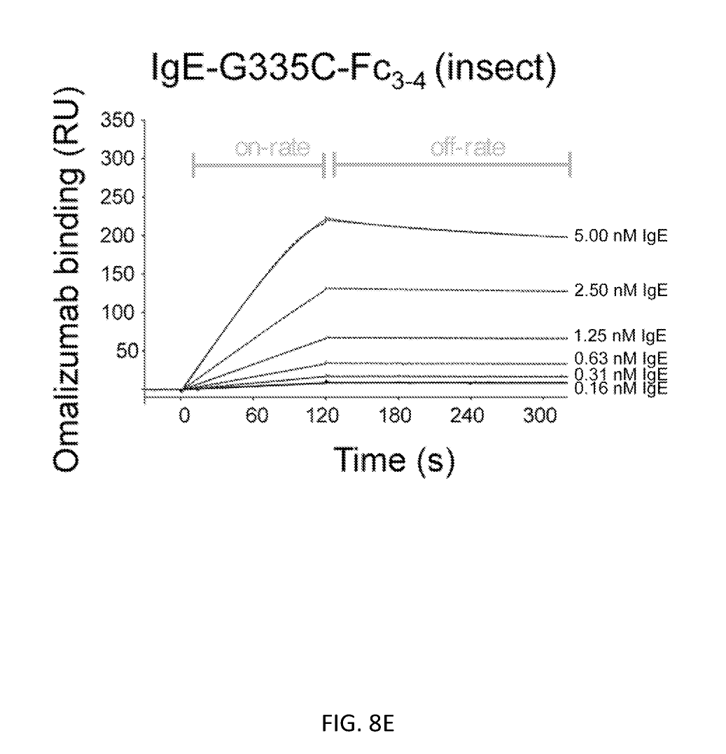

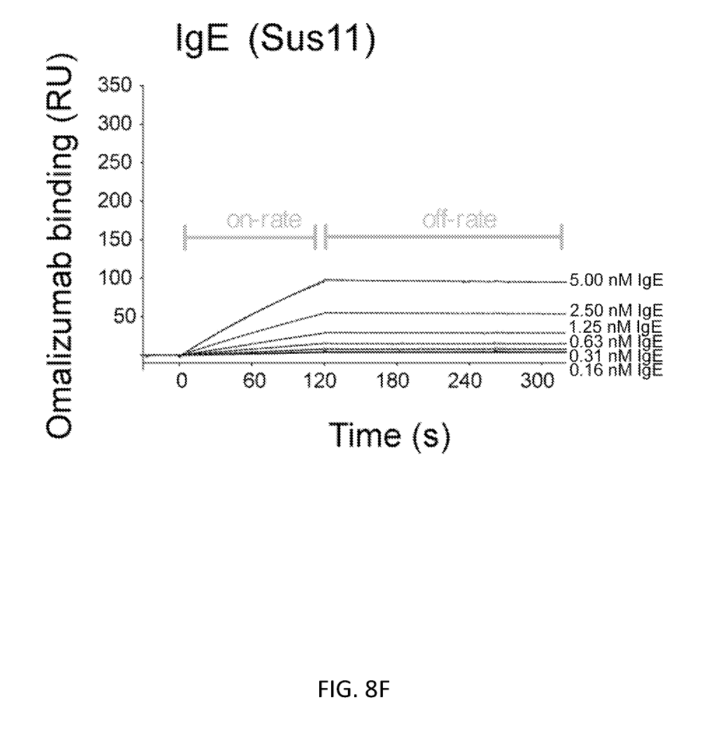

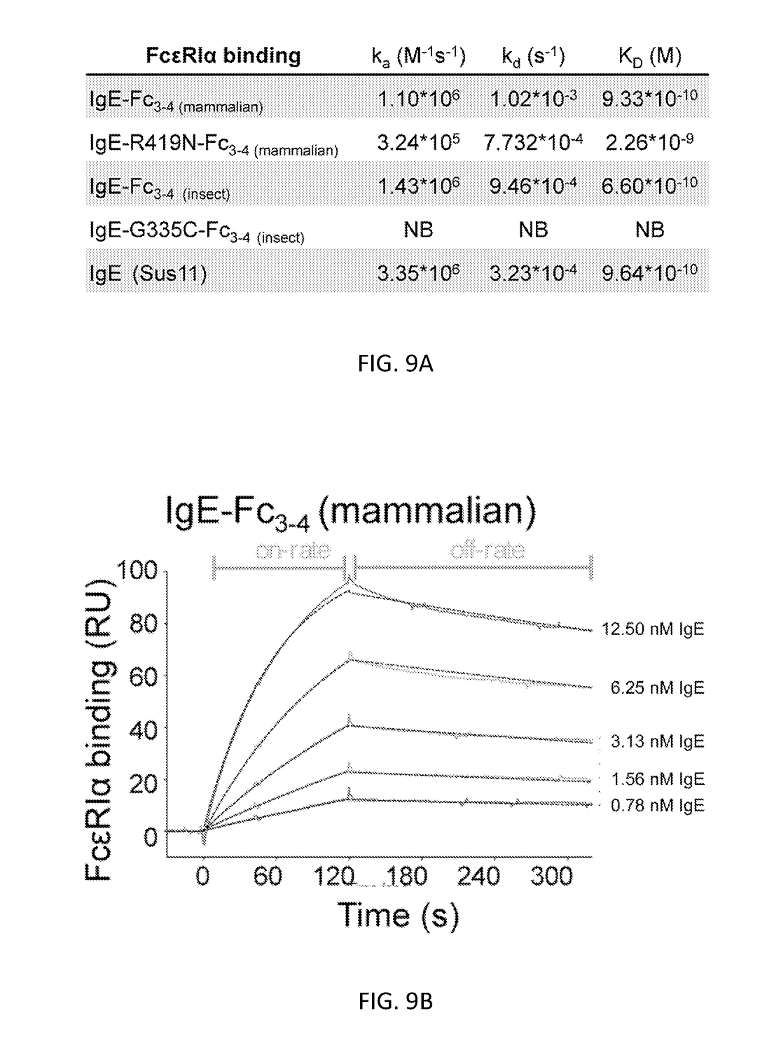

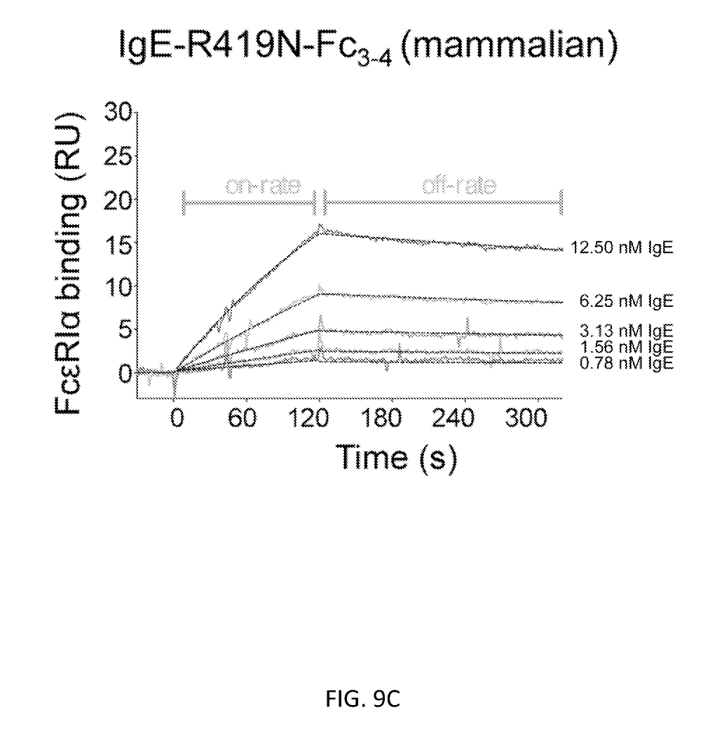

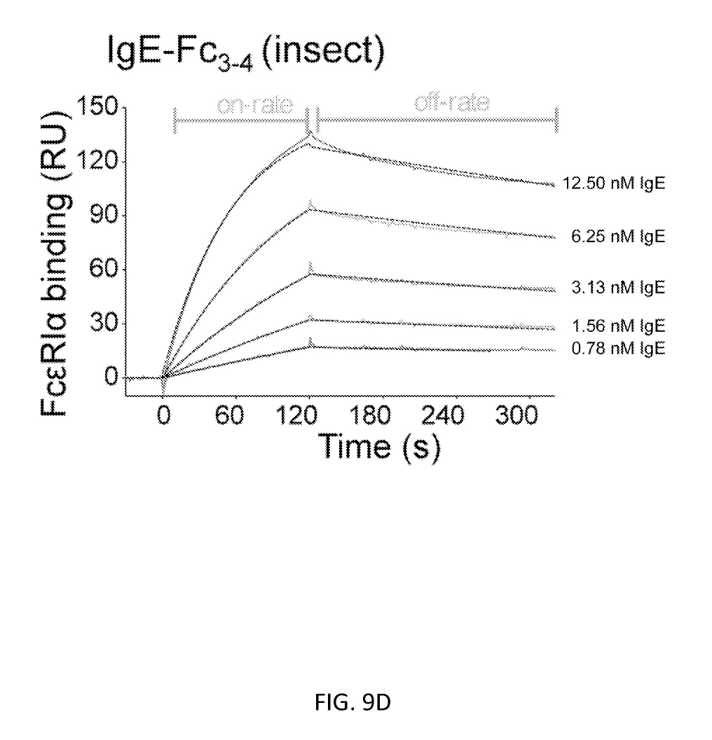

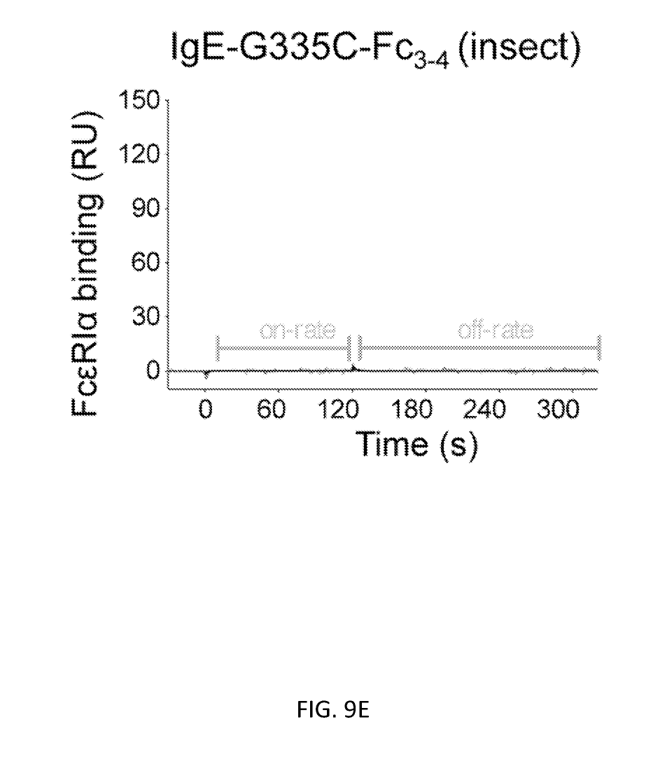

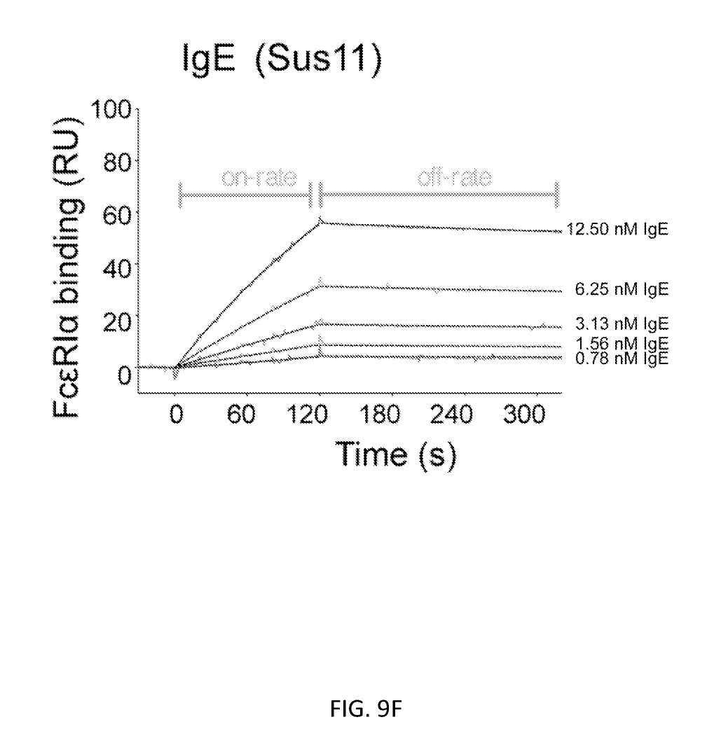

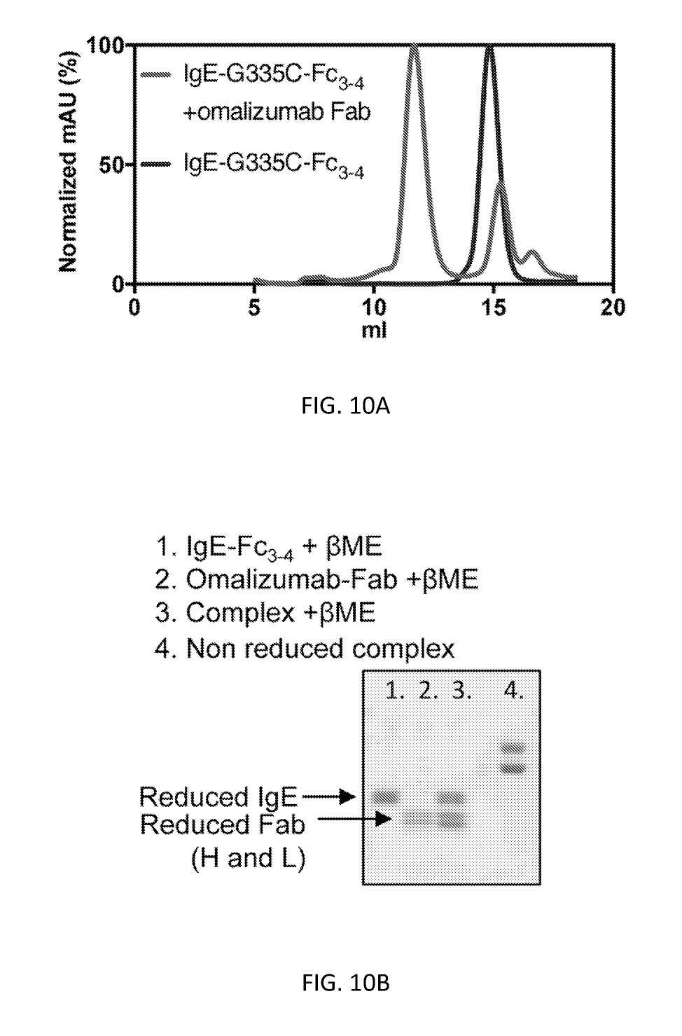

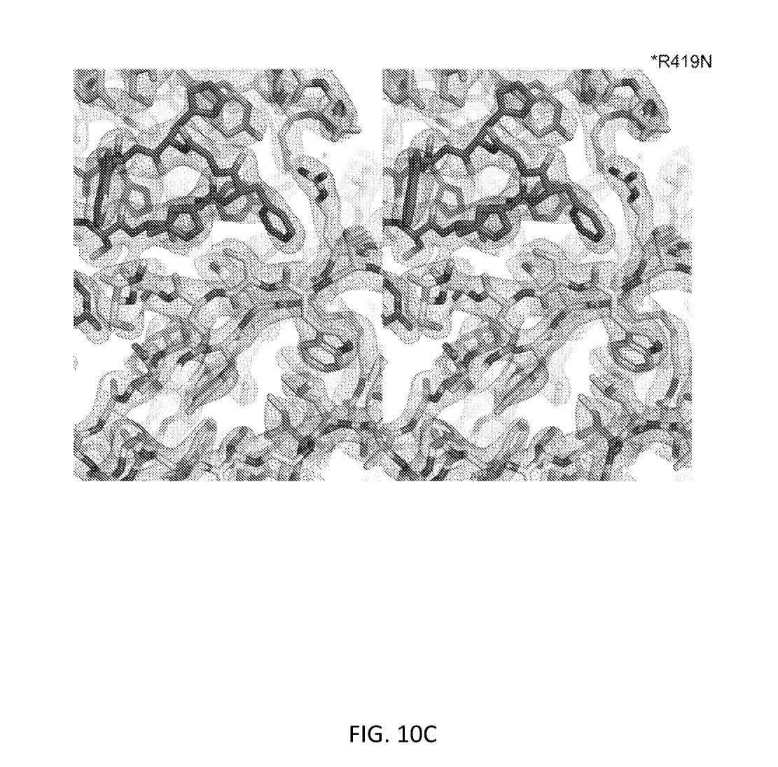

We previously described an IgE-Fc₃₋₄ mutant (IgE-G335C-Fc₃₋₄), which contains an engineered disulfide bond at position 335 that traps the IgEFc₃₋₄ domain in a closed conformation with reduced conformational flexibility (FIG. 1B, Wurzburg et al. (2012) J. Biol. Chem. 287:36251-36257). This variant retains high-affinity binding to omalizumab, but not FcεRIα (FIGS. 8 and 9). Employing this restrained IgE-Fc₃₋₄ variant, we crystallized the IgE-G335C-Fc₃₋₄:omalizumab-Fab complex (subsequently referred to as the IgE:omalizumab complex). The purified complex (FIGS. 10A, 10B) crystalized in the P21 space group, diffracted X-rays to 2.5 Å (Table 1), and the structure was solved by molecular replacement using the existing IgE-G335C-Fc₃₋₄ and omalizumab-Fab structures (Wurzburg et al. (2012), supra; Jensen et al. (2015) Acta Crystallogr. F Struct. Biol. Commun. 71:419-426). The asymmetric unit contains two copies of IgE-G335C-Fc₃₋₄, and four copies of the omalizumab-Fab, providing four copies of the IgE:omalizumab interface related by non-crystallographic symmetry (NCS). Side-chain electron density was well resolved throughout the IgE:omalizumab interface (FIG. 10C).

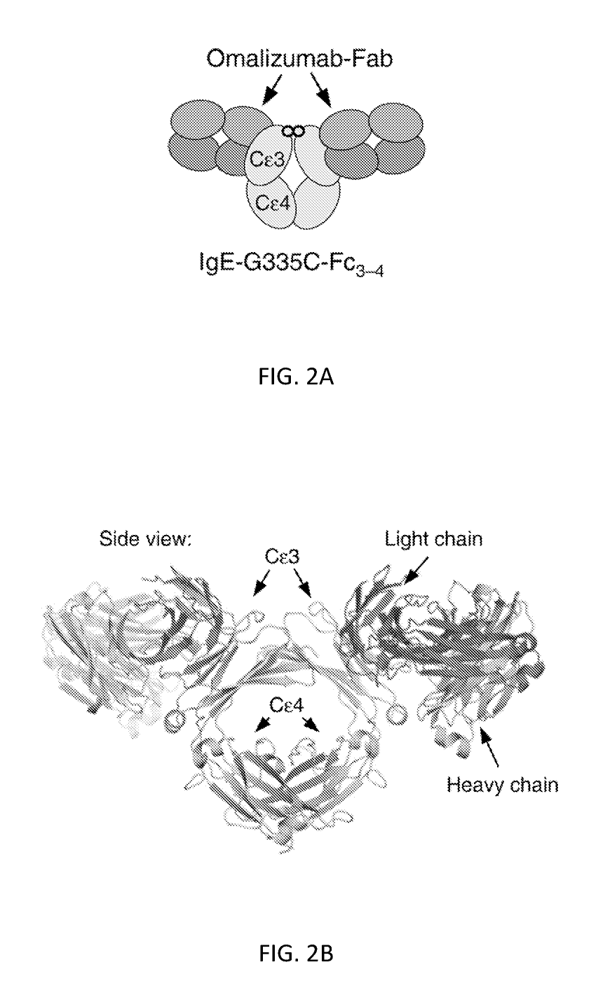

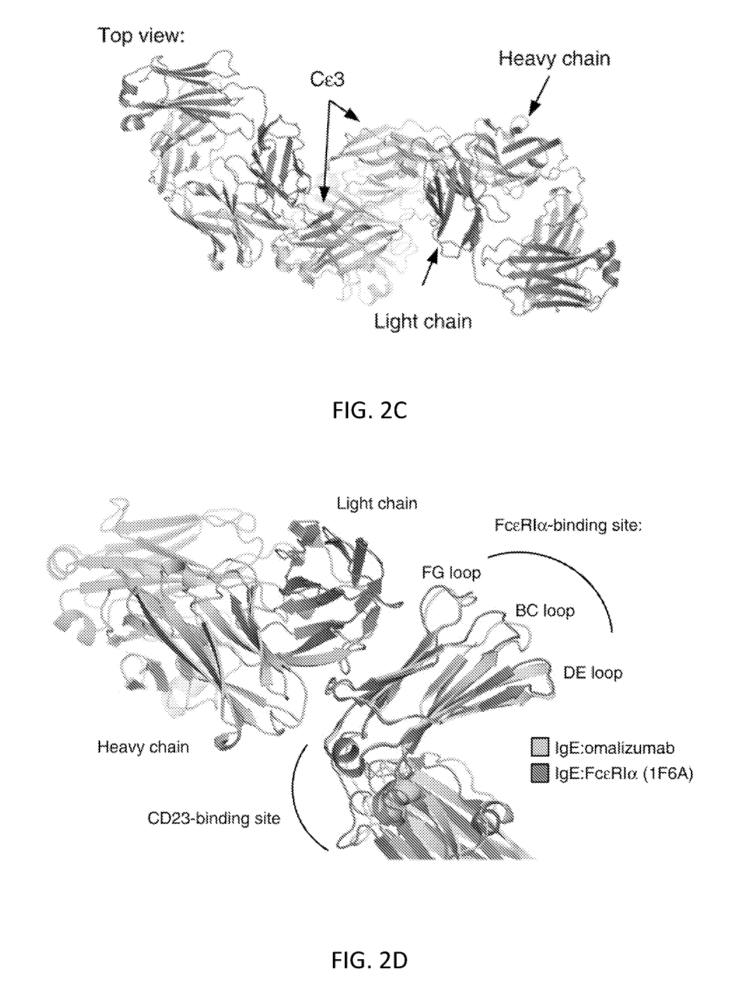

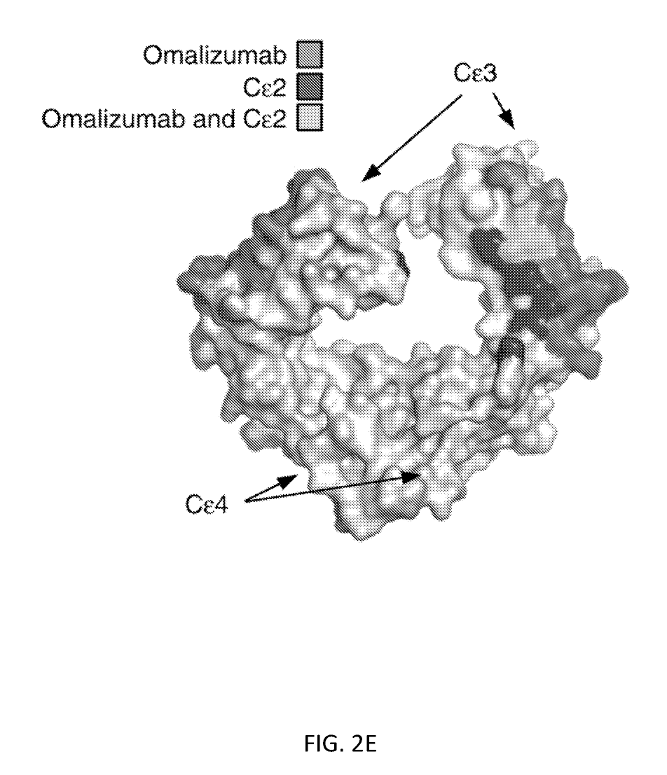

Analysis of the structure revealed that the omalizumab-Fab approaches perpendicularly to the IgE Cε3 domain, in contrast to recently proposed models (Wright et al. (2015) Sci. Rep. 5:11581), and contacts symmetric binding sites on the face of the two Cε3 domains of the IgE dimer (FIGS. 2A-2C). These contacts are predominantly formed between the omalizumab-Fab and Cε3 β-sheet residues below the FcεRIα-binding loops (FG, BC and DE; FIG. 2D). Structural alignment of the IgE:FcεRIα complex (Garman et al., supra) with the IgE:omalizumab complex shows that the binding loops are only slightly perturbed within the IgE:omalizumab complex at receptor site 2 (FIG. 2D); thus, omalizumab does not appear to inhibit IgE:FcεRIα interactions by distorting the FcεRIα-binding site. Instead, the omalizumab-Fab is positioned between the binding sites of both FcεRI and CD23, blocking interactions with both receptors, with the heavy chain proximal to the CD23 site, and the light chain proximal to the FcεRIα-binding sites (FIG. 2D). Of note, both the position of the omalizumab epitope on the face of Cε3, and the perpendicular binding orientation of the omalizumab Fab, suggest that omalizumab could approach and target a preformed IgE-FcεRI complex.

Impact of IgE-Fc Conformation on Omalizumab Binding.

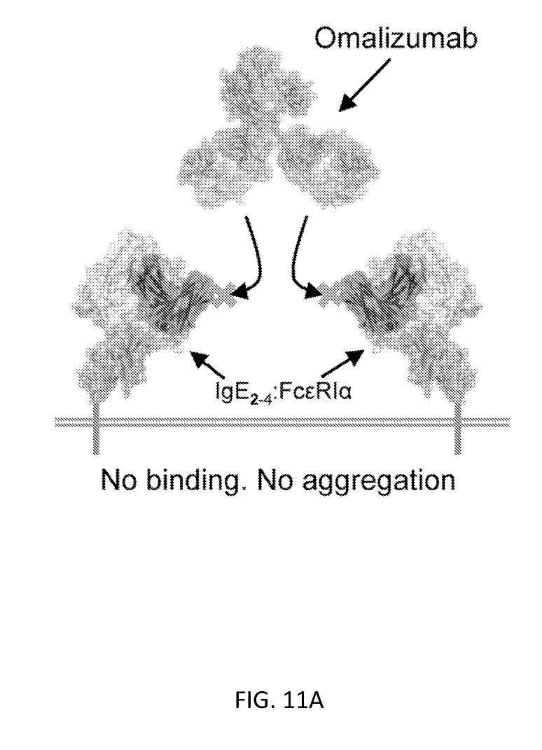

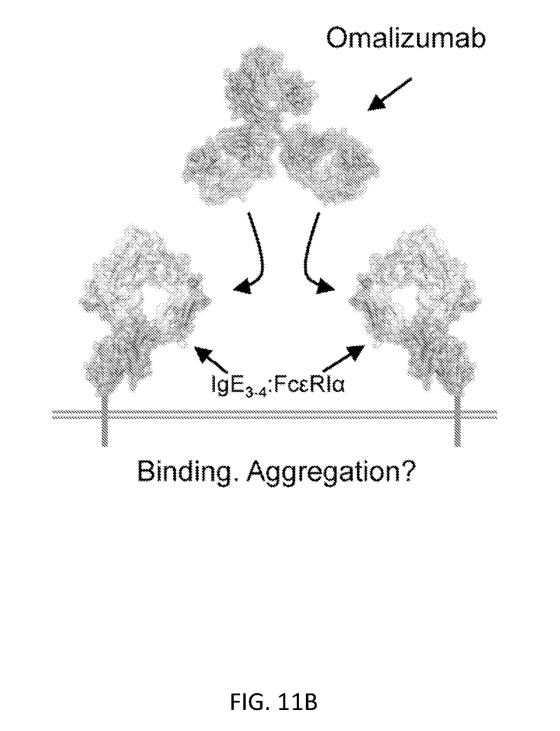

We previously demonstrated that omalizumab can bind preformed IgE-Fc₃₋₄:FcεRIα complexes, but not full-length IgE:FcεRIα complexes (Eggel et al., supra). Therefore, we hypothesized that the Cε2 domains of full-length IgE in the IgE:FcεRIα complex obscure an omalizumab-binding site that is exposed in the IgE-Fc₃₋₄:FcεRIα complex. As predicted, alignment of the structures of IgE:omalizumab and IgE-Fc₂₄:FcεRIα complexes demonstrates that the Cε2 domains overlap extensively with an otherwise exposed omalizumab-binding epitope in the IgE-Fc₃₋₄:FcεRIα structure (FIG. 2E). Therefore, omalizumab's specificity for free IgE is not solely determined by FcεRIα competition, but also by IgE-conformation-dependent masking of its own second omalizumab epitope. These observations suggest that fragmented IgE molecules lacking Cε2 could be aggregated by omalizumab, leading to basophil activation (FIG. 11); however, we have not observed this in human basophils or mast cells (Eggel et al., supra).

Given that the crystal structure lacks the Cε2 domain, it cannot account for possible contributions of the Cε2 to omalizumab:IgE interactions. Therefore, we also compared the binding kinetics of full-length IgE (clone Sus11) to that of the IgE-Fc₃₋₄ fragments. We hypothesized that the kₐ of full-length IgE might be slower than that of IgE-Fc₃₋₄ because the Cε2 domains obscure one of the two symmetric omalizumab epitopes (FIG. 1), and, as expected, the kₐ of full-length IgE was 15-30-fold lower as compared with the IgE-Fc₃₋₄ fragments tested (FIG. 8). Surprisingly, the k_{d} of the full-length IgE was also 4.5-10.9-fold slower than any of the three IgE-Fc₃₋₄ fragments tested (FIG. 8). This results in only minor differences in the overall equilibrium K_{d}(˜3.5-fold) between intact IgE and the IgE-Fc₃₋₄ fragments.

The IgE Cε3 domains also show significant conformational flexibility, adopting closed and open states relative to the Cε4 domains that are associated with CD23 and FcεRI binding, respectively. The potential impact of these conformational changes on omalizumab binding has not been fully assessed. The localization of the omalizumab epitope on the face of the Cε3 domain suggested that omalizumab would interact equally well with both open and closed forms of the IgE-Fc. To examine this possibility, we compared the binding kinetics of omalizumab with wild-type IgE-Fc₃₋₄, the IgE-G335C-Fc3-4 mutant (locked in the closed conformational state (Wurzburg et al. (2012) J. Biol. Chem. 287:36251-36257)) and with IgE-Fc₃₋₄ bound to FcεRIα (stabilized in the open state by receptor binding). IgE-G335C-Fc₃₋₄ exhibited similar kinetics in omalizumab-binding studies as wild-type IgE-Fc₃₋₄ (FIG. 8) but was unable to bind FcεRIα (FIG. 9). These data suggest that omalizumab can bind closed conformations of IgE efficiently, and indicate that the crystal structure reflects the normal binding mode of omalizumab for IgE-Fc₃₋₄.

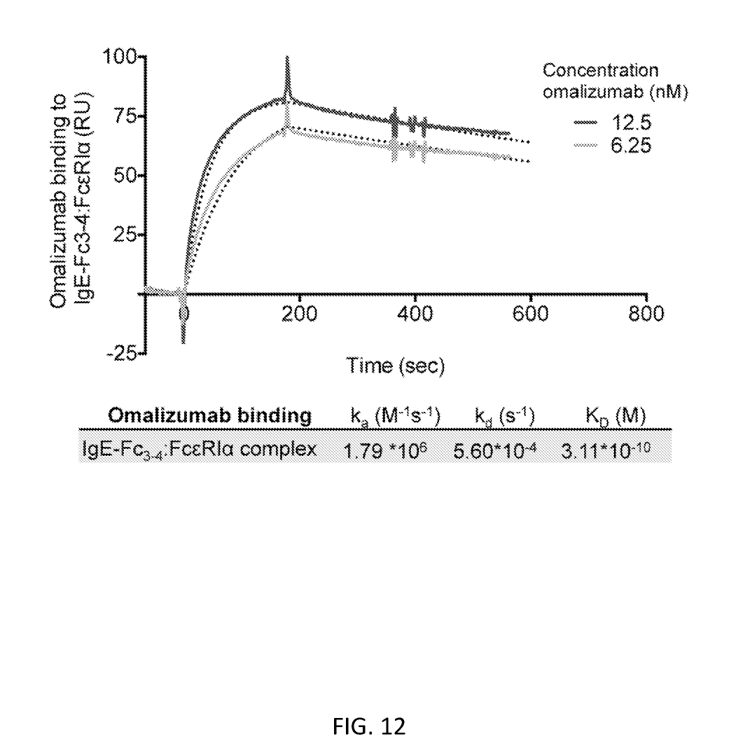

No mutations have been identified that stabilize the open IgE⁻Fc₃₋₄ conformational state, making studies of the omalizumab interaction with this state more challenging. However, omalizumab binds to IgE-Fc₃₋₄:FcεRIα complexes (Eggel et al., supra), and our structural analysis demonstrates that one of the two Cε3 domain epitopes is fully accessible to omalizumab. Therefore, to examine the potential impact of the open Cε3 domain conformation on omalizumab binding, we measured the kinetics of omalizumab binding to preformed IgE-Fc₃₋₄:FcεRIα complexes. This analysis revealed an association rate constant (kₐ) for omalizumab with IgE-Fc₃₋₄ bound to FcεRIα complexes that was closer to full-length IgE alone and slower than unbound IgE-Fc₃₋₄. The similarity in association rates between full-length IgE and IgE-Fc₃₋₄:FcεRIα complexes may be in part because of the fact that each binding partner contains a single exposed Cε3 domain (FIG. 12). The dissociation rate constant (k_{d}) for the omalizumab:IgE-Fc₃₋₄:FcεRIα complexes was also similar to that of the measured rates for IgE:FcεRIα or omalizumab:IgE complexes (FIGS. 8 and 9), consistent with the dissociation of either of these two interfaces during measurement.

Together, these kinetic data demonstrate that the IgE-Fc Cε3 conformations have little or no impact on omalizumab binding and that, in free IgE, the Cε2 domain may alter the kinetics of omalizumab binding with a small effect on the affinity of the interaction. These data support the structural observation that omalizumab:IgE interactions are primarily mediated by a stable epitope contained in the Cε3 domain. These kinetic data also highlight the critical role of the Cε2 domains in the intact receptor-bound IgE, which are required to mask an omalizumab epitope that is not directly blocked by the FcεRI itself.

The Omalizumab Epitope.

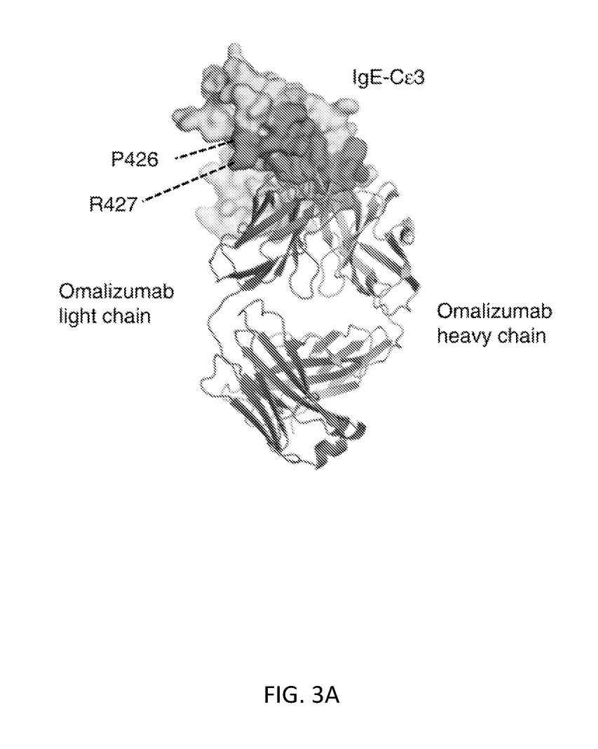

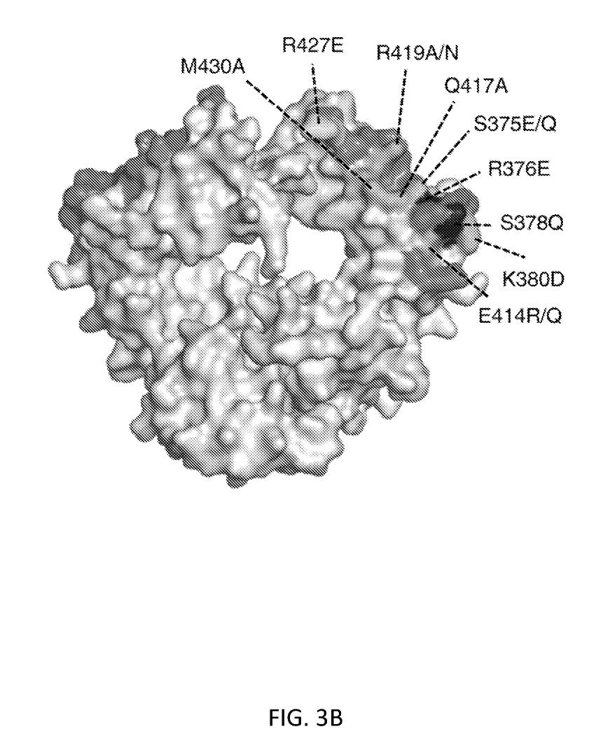

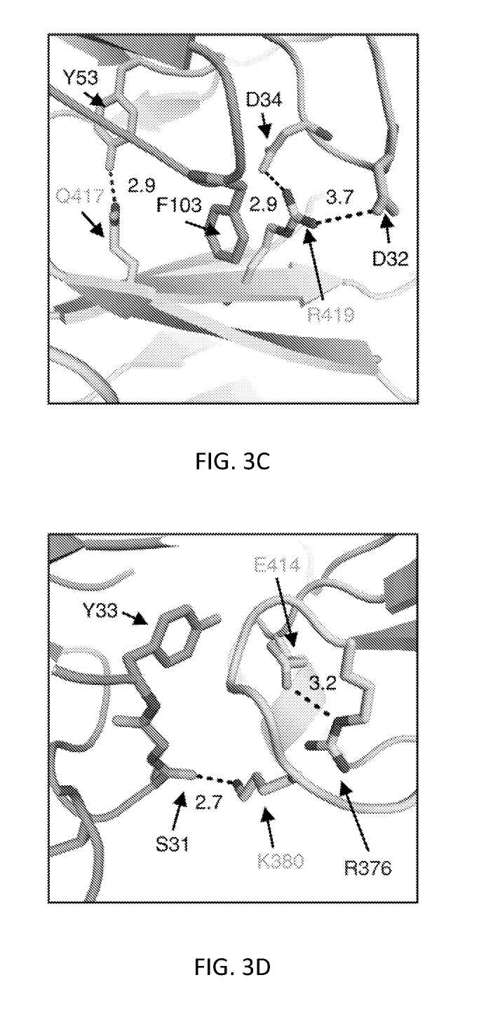

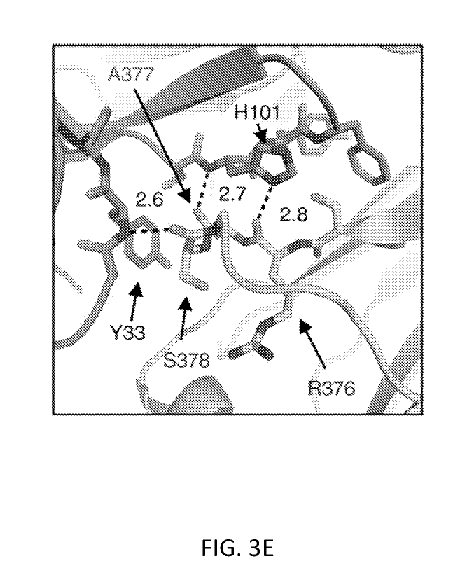

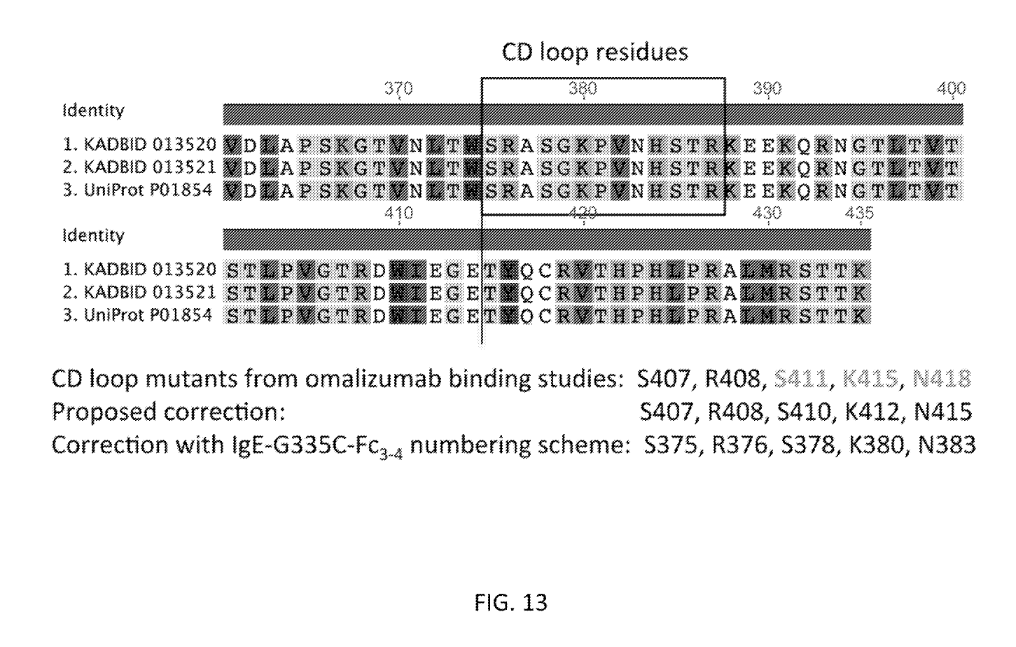

To establish which residues fall within the omalizumab epitope on IgE, we analyzed the interfaces of the omalizumab:IgE complex with PISA (Krissinel et al. (2007) J. Mol. Biol. 372:774-797). All NCS copies shared the majority of contacts, which extend along the length of the Cε3 domain, involve 23 IgE residues and bury ˜725 Å² of surface area on IgE (FIG. 3A). Previously identified IgE mutants that inhibit omalizumab binding, studied in an intact IgE heavy chain, correspond well with the binding interface observed in the crystal structure (FIG. 3B, Presta et al., supra). After correcting for different numbering schemes and IgE sequences from prior studies (FIG. 13), all residues implicated in omalizumab: IgE binding are within the predicted omalizumab:IgE interface, and many participate in hydrogen bonds (R376, S378, K380, Q417) or salt bridges (R419) predicted by the crystal structure (FIGS. 3C-3E). Notably, IgE E414, a residue implicated in omalizumab-binding studies with the mutants E414R/Q, appears to form an intrachain salt bridge with IgE R376 (FIG. 3D) and may be essential for stabilizing the conformation of the adjacent omalizumab-binding residues (FIGS. 3D, 3E).

The omalizumab complementarity-determining region (CDR) loops, with the exception of the light chain CDR3 loop, contact IgE. CDR residues previously shown to be required for omalizumab binding either contact IgE (light chain D32 in CDR1 (FIG. 3C) and heavy chain H101 in CDR3 (FIG. 3E)) or form interactions with neighboring CDR loops (heavy chain CDR3 H105 and H107) (Presta et al., supra). The complex contains five hydrogen bonds distributed throughout the omalizumab:IgE interface and two salt bridges between light chain residues D32 and D34 and IgE R419 (FIGS. 3C-3E). The heavy chain CDR3, which shows the most extensive conformational change from the unbound omalizumab:Fab structure (PDB ID: 4X7S), also contains three aromatic side chains that contact IgE (Y102, H101, F103; FIG. 3E). Outside of the omalizumab heavy chain CDR3, four additional aromatic side chains contact IgE: Y33 in the heavy chain CDR1, Y36 in the light chain CDR1 and Y53 and Y57 in the light chain CDR1. Therefore, it appears that a network of hydrogen bonds, salt bridges and extensive hydrophobic interactions facilitate omalizumab:IgE interactions.

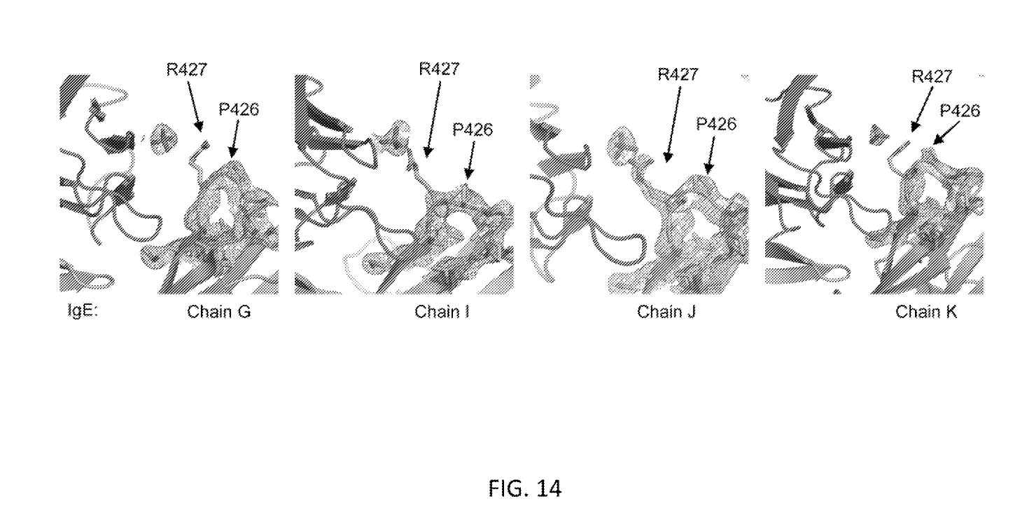

The IgE residues interacting with omalizumab CDRs are largely distinct from those that engage FcεRIα (Presta et al, supra). IgE residues P426 and R427 are the only minor overlapping portions of the omalizumab:IgE-Fc3-4 and FcεRIα:IgE-Fc₃₋₄ interfaces as calculated with the PISA analysis (FIG. 3A). Each residue is unique to site 2 of the FcεRIα:IgE complex. The residues are adjacent to the omalizumab light chain framework region (FIG. 3A and FIG. 14) and are peripheral to the omalizumab:IgE interface. Only one NCS-related IgE chain has well-resolved electron density for R427, while the remaining copies do not (FIG. 14). Within this chain neither R427 nor P426 make contacts (<4 Å) directly with omalizumab; however, R427 indirectly interacts with the light chain framework through a sulfate ion (FIG. 14). Mutations at R427 (R427E) lead to a minor reduction in omalizumab binding (25-56%), suggesting that this residue can affect omalizumab:IgE interactions (Presta et al, supra). In contrast, the R427E mutation substantially reduced FcεRIα binding to IgE, while another mutant series that contained a R427A mutation only partially reduced FcεRIα binding (Presta et al., supra). Although omalizumab may directly compete with FcεRIα for IgE residues, the extent of direct competition and binding site overlap involves at most two amino acids.

The Structural Basis of FcεRI and CD23 Competition.

Omalizumab inhibition of FcεRIα and CD23 binding could arise from contributions of multiple structural mechanisms, including direct competition for receptor-binding residues on IgE, steric clashes caused by physical overlap of omalizumab and IgE receptors and potential omalizumab-induced conformational changes in IgE. The kinetic and structural data suggest that omalizumab does not induce conformational changes in FcεRIα-binding loops or in the relative positions of the Cε3 domains that could affect receptor binding. Omalizumab also shows minimal overlap with FcεRIα-binding residues; however, physical overlap between the bound omalizumab Fab and FcεRIα could be substantial and critical to omalizumab activity.

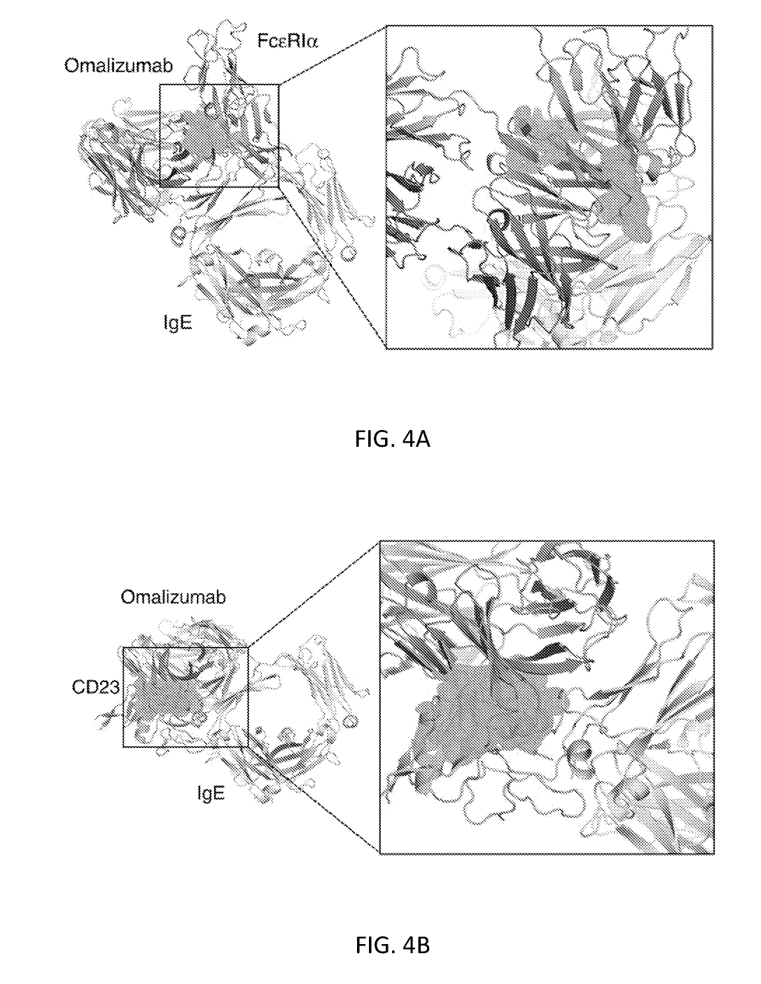

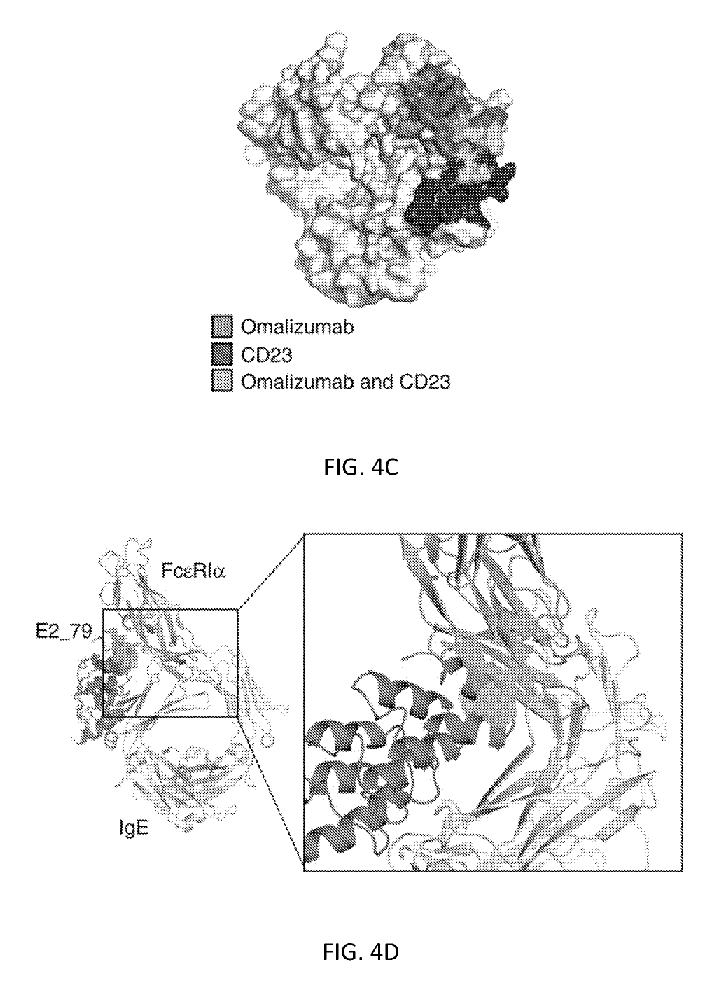

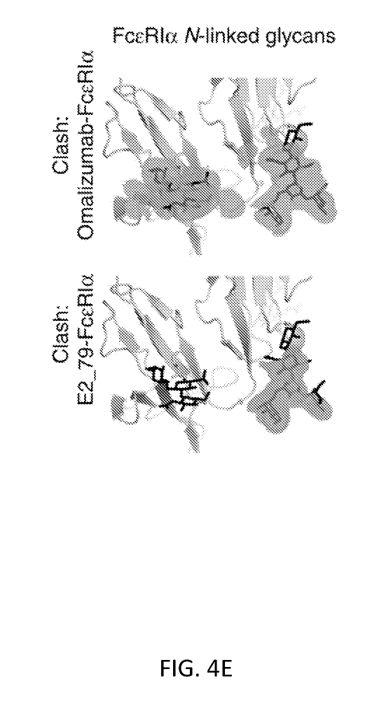

To gain quantitative insight into the contribution of inhibitor overlap in blocking IgE interactions with FcεRIα and CD23, we calculated the theoretical volumes of atomic overlap between omalizumab and its two IgE receptors. First, we performed a structural alignment of the Cε3 domain of the IgE:omalizumab complex with the Cε3 domain of the IgE:FcεRIα complex at sites 1 and 2 (FIG. 4A). This alignment strategy accounts for the variability of open and closed IgE conformations observed across IgE crystal structures (Dhaliwal et al., supra; Wurzburg et al. (2012) J. Biol. Chem. 287:36251-36257; Wurzburg et al. (2009) J. Mol. Biol. 393:176-190). We then calculated the volume of atomic overlap between the superimposed omalizumab and FcεRIα proteins. This analysis revealed that, for the omalizumab-binding site proximal to FcεRIα binding site 2, there are significant steric clashes between the antibody light chain and both domains of the FcεRIα receptor (FIG. 4A), while no clashes exist at site 1. These structural data indicate that omalizumab's mechanism of FcεRIα inhibition involves substantial steric conflict with the receptor at site 2, while direct competition for FcεRIα-binding residues is limited.

Omalizumab has also been shown to inhibit the binding of CD23 (Cohen et al. (2014) MAbs 6:756-764). Both substantial steric overlap between omalizumab and CD23, and direct competition for IgE-binding residues by the omalizumab heavy chain, contribute to omalizumab inhibition of CD23 binding (FIGS. 4B, 4C). The degree of steric overlap of omalizumab with CD23 is significantly greater than that observed for FcεRIα (FIG. 4B). Binding-site comparisons also demonstrate a more extensive overlap between IgE residues that engage omalizumab and CD23 in their respective complexes as compared with FcεRIα (FIG. 4C).

Steric Overlap and Inhibitor-Induced FcεRIα a Complex Dissociation.

We recently described a class of IgE inhibitors derived from Designed Ankyrin Repeat Protein (DARPᵢₙ) libraries, capable of disrupting IgE:FcεRI complexes (Kim et al. (2012) Nature 491:613-617; Eggel et al. (2014) J. Allergy Clin. Immunol. 133:1709-1719). These agents target preformed IgE: FcεRIα complexes found on mast cells and basophils and accelerate the dissociation rate constant to release free IgE. The activated release of IgE on the surface of effector cells might prove beneficial in treating acute allergic reactions and enhance the clearance of allergen-specific IgE during anti-IgE therapy by targeting both cellular and serum pools of IgE simultaneously. We have demonstrated that a bivalent DARPᵢₙ (bi53_79) containing a non-competitive IgE-binding domain and a disruptive competitor domain dissociates complexes with greater efficiency in vitro and shows greater potency than omalizumab in blocking passive cutaneous anaphylaxis in mice bearing the human FcεRI receptor (Eggel et al., supra). To our surprise, we also observed that omalizumab is not strictly a competitive inhibitor of IgE:FcεRIα interactions, but at higher concentrations it is also capable of targeting and disrupting IgE:FcεRIα complexes (Eggel et al., supra). We have published the crystal structure of the DARPᵢₙ-based inhibitor E2_79 (Kim et al., supra), which is able to accelerate the dissociation of FcεRIα complexes at concentrations B3,000× above the E2_79:IgE K_{D} (Kim et al., supra). In contrast, omalizumab shows an ability to disrupt preformed FcεRIα complexes at concentrations that are much higher (˜1,000,000-fold greater) than the omalizumab:IgE K_{D} (Eggel et al., supra), indicating that it is much less efficient at the process of binding to and dissociating these preformed complexes. We hypothesized that this difference in disruptive capability was related to the binding-site locations for E2_79 and omalizumab on IgE and the level of atomic overlap between each inhibitor and receptor. We therefore compared the structure of both the E2_79:IgE-G335C-Fc₃₋₄ complex (E2_79:IgE) and the omalizumab:IgE complex.

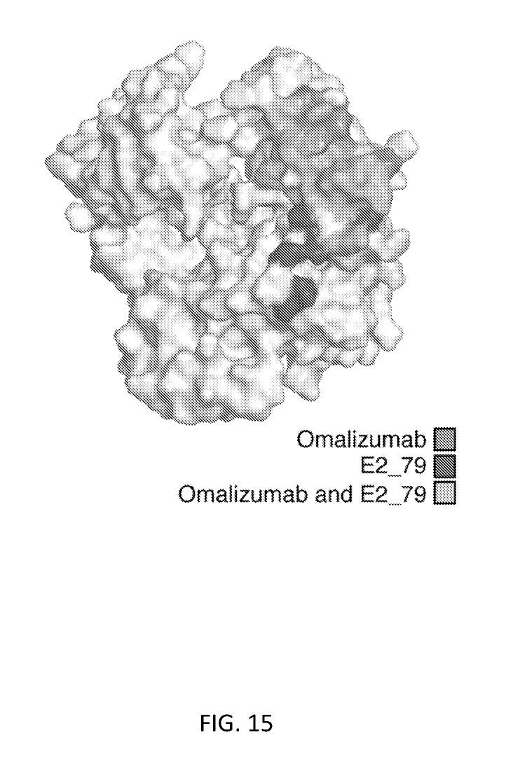

The structure of the E2_79:IgE complex demonstrated that the E2_79-binding sites do not overlap with FcεRIα-binding residues, while omalizumab exhibits only minor peripheral interactions with two FcεRIα-binding residues (Kim et al., supra). Instead, similar to omalizumab, E2_79 exhibited steric conflicts with FcεRIα in aligned structures of the complexes. Given that both agents can disrupt preformed IgE:FcεRIα complexes (Eggel et al., supra), we sought to quantitatively compare their relevant steric clashes with FcεRIα when bound to IgE, by computing the predicted volume of atom-atom overlap of each inhibitor with FcεRIα in aligned complex structures. This analysis reveals that omalizumab has roughly three times the volume of atomic overlap with FcεRIα compared with E2_79 (omalizumab and FcεRIα=1,183 Å³ versus E2_79 and FcεRIα=401 Å³). The omalizumab steric conflicts extend along the length of the omalizumab light chain and FcεRIα N-terminal domain, while the E2_79 steric conflicts are more localized near the IgE:FcεRIα interface (FIGS. 4A and 4D, Pettersen et al. (2004) J. Comput. Chem. 25:1605-1612). Since the E2_79 and omalizumab-binding sites are substantially overlapping on the IgE-Fc (FIG. 15), this large difference in steric overlap with FcεRIα stands out as a prominent structural feature that correlates with the relative disruptive activities of these inhibitors. Conformational dynamics in the IgE:FcεRIα complex may transiently allow E2_79 association and subsequent acceleration of FcεRIα dissociation (Kim et al., supra). Given the significantly larger region of steric conflicts observed between omalizumab and FcεRIα, conformational states of the IgE:FcεRIα that could accommodate omalizumab association may simply be less accessible and/or occur with lower frequency, explaining its lower activity. A significant fraction of the steric clashes with both inhibitors occur between the protein backbone of omalizumab or E2_79 and carbohydrate groups on FcεRIα (FIG. 4E). These carbohydrate groups likely explore a wider range of conformations, and may help facilitate the association of these inhibitors to preformed IgE:FcεRIα complexes.

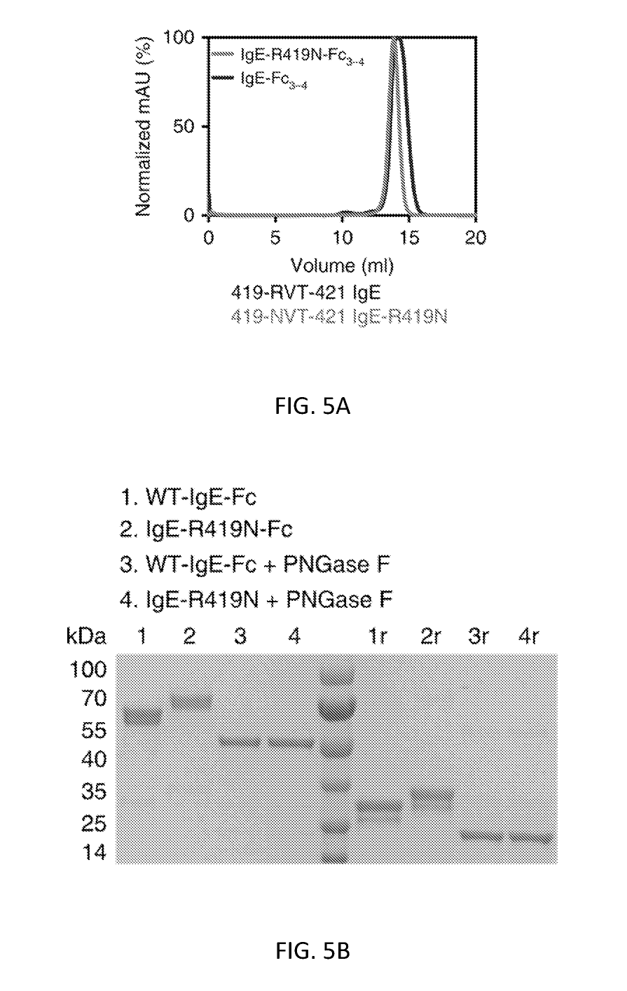

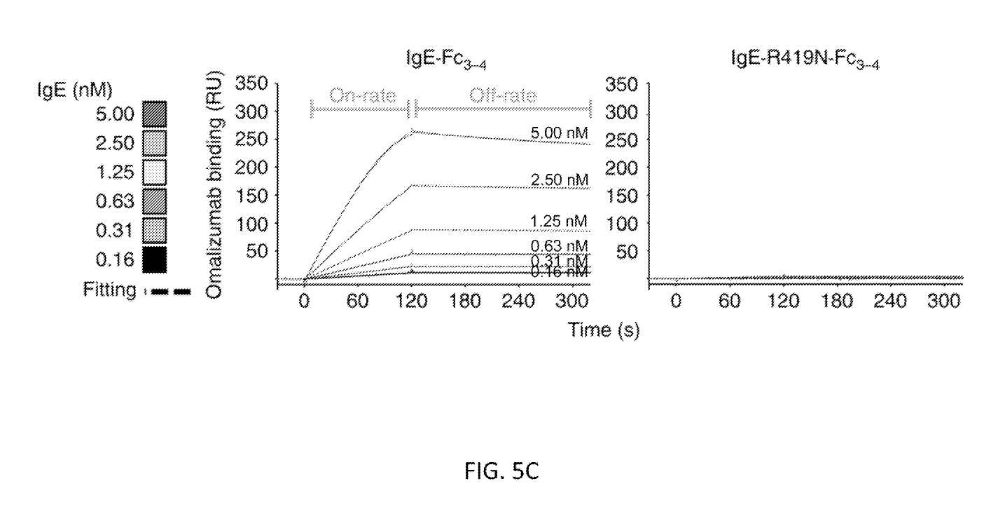

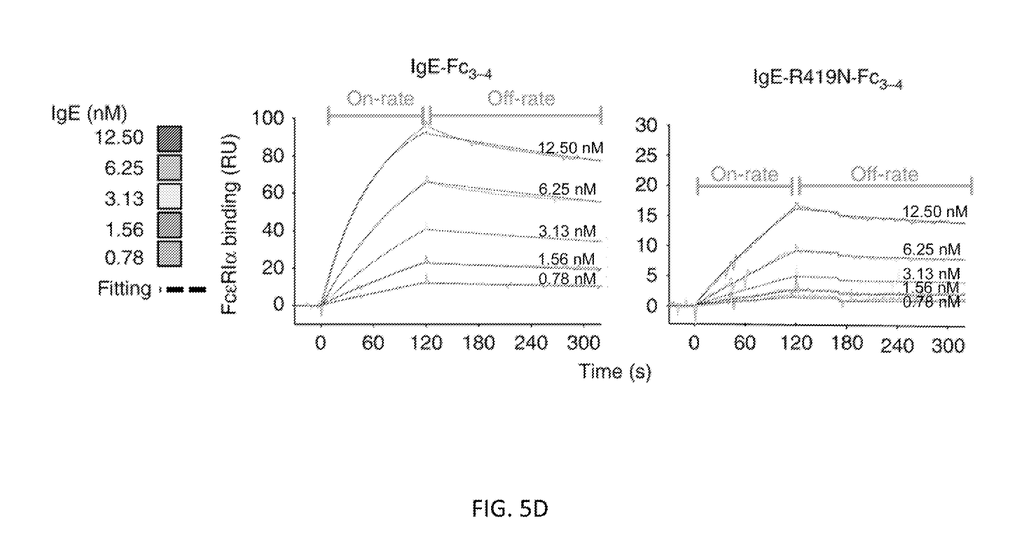

A single IgE mutation prevents omalizumab binding. To further validate observations from the crystal structure, we produced an additional IgE-Fc₃₋₄ mutant. IgE residue R419 lies at the interface of the IgE:omalizumab complex, forming contacts with both light and heavy chain CDR loops (FIG. 3C) and participating in salt bridges. Mutation of R419 to an asparagine (R419N) introduces the glycosylation consensus sequence—asparagine valine threonine (NVT) (FIG. 5A). We hypothesized that by mutating this residue we would abolish omalizumab binding by introducing a glycosylation site at the core of the omalizumab epitope.

The R419N mutation induced a shift in the mass of recombinant IgE-Fc₃₋₄ as assessed by SDS-PAGE and gel filtration, consistent with the introduction of an additional N-linked glycan (FIGS. 5A, 5B). The IgE-Fc₃₋₄ contains two N-linked glycosylation sites, N371 and N394 (Nettleton et al. (1995) Int. Arch. Allergy Immunol. 107:328-329; Shade et al. (2015) J. Exp. Med. 212:457-467). We have found in recombinant preparations of IgE-Fc₃₋₄ that glycosylation at N371 is heterogeneous and leads to a minor band in purified material (FIG. 5B). Both major and minor species of IgE-R419N-Fc₃₋₄ remain in similar proportions to wild-type IgE-Fc₃₋₄ species, with similar mobility shifts in SDS-PAGE. Furthermore, the shift in apparent mass for IgE-R419N-Fc3-4 relative to wild-type IgE-Fc3-4 was lost on digestion with PNGaseF, confirming that it is caused by N-linked glycosylation (FIG. 5B). This additional glycosylation in the middle of the omalizumab epitope completely abolishes omalizumab binding, but only slightly perturbs IgE-R419N-Fc₃₋₄ binding to FcεRIα as assessed by surface plasmon resonance (SPR; FIGS. 5C, 5D and summary data FIGS. 8 and 9). Taken together, these data demonstrate that the R419N mutation introduces a novel glycosylation site to yield an omalizumab-resistant IgE-Fc₃₋₄ variant.

Exchange of IgE on human basophils. The paradigm of using anti-IgE treatment to deplete free IgE (both allergic and non-allergic species) has proven successful for controlling allergic diseases. During omalizumab therapy, omalizumab neutralizes free serum IgE and slowly decreases surface levels of IgE on allergic effector cells. Therefore, it is impossible to simultaneously replace depleted allergen-reactive species with benign IgE species because of the requirement for continued excess omalizumab to be present during treatment. Since IgE-dependent homeostatic regulatory pathways could potentially counteract anti-IgE therapies, we sought to explore the possibility of replacing rather than removing patient IgE. We hypothesized that omalizumab-resistant IgE variants or fragments could be used in combination with omalizumab to effectively exchange the native IgE on human cells bearing FcεRI or CD23.

Over the course of omalizumab treatment, free IgE levels and IgE surface levels on basophils decline relatively rapidly within days (Arm et al. (2014) Clin. Exp. Allergy 44:1371-1385), while mast cell IgE levels persist for significantly longer (Beck et al. (2004) J. Allergy Clin. Immunol. 114:527-530). The rapid decline of basophil-associated IgE may in part be driven kinetically by basophil turnover in vivo (MacGlashan et al. (2015) J. Allergy Clin. Immunol. 135:294-295). However, within the experimental timeframe for our ex vivo experiments with human whole blood (24-48 hours), we did not observe significant loss of cell surface IgE at doses corresponding to the mean omalizumab serum concentrations from clinical trials (Korn et al. (2012) Respir Med. 106:1494-1500). Therefore, we employed supraphysiologic doses of omalizumab (25 μM) to enhance the removal of IgE as described previously (Eggel et al., supra). These doses were used here to accelerate the loss of cell-bound IgE to facilitate these exchange experiments. Nonetheless, lower therapeutic doses of omalizumab are sufficient to capture free IgE and reduce cell surface levels of IgE over time, which is the basis of omalizumab's therapeutic effect.

To track the addition, removal and exchange of IgE species, we employed biotinylated IgE-Fc₃₋₄ or omalizumab-resistant IgE-R419N-Fc₃₋₄, and the JW8 human/mouse chimeric IgE (composed of a human IgE heavy chain and a mouse-l-light chain). The JW8 mouse-l-light chain allowed us to track IgE reloading on stripped basophils in parallel with the biotinylated IgE⁻Fc₃₋₄ proteins. We ensured that all antibody reagents used in staining carried the mouse-k-light chain, or were rat antibodies, to avoid nonspecific binding from the anti-mouse-l-light-chain antibody used to track JW8-IgE.

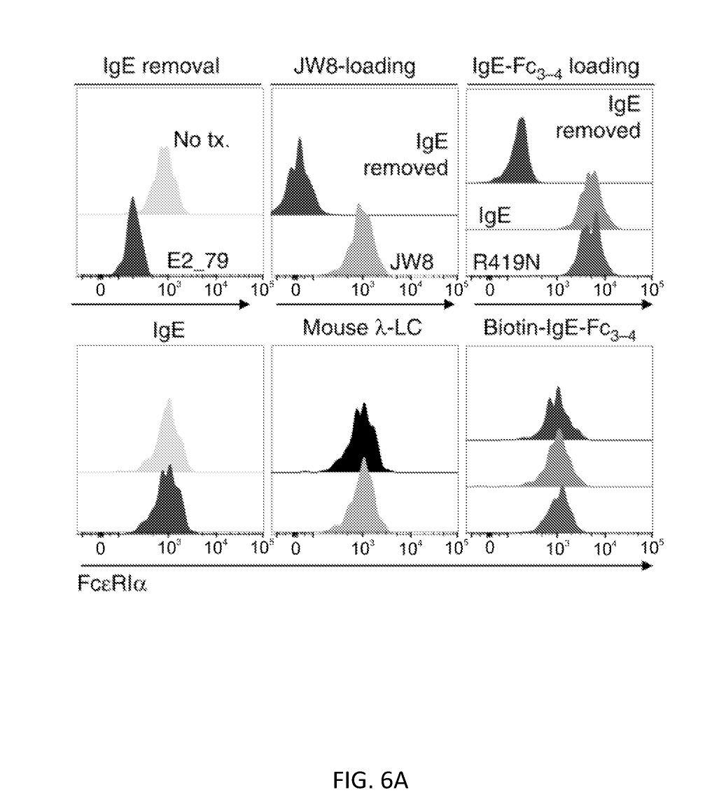

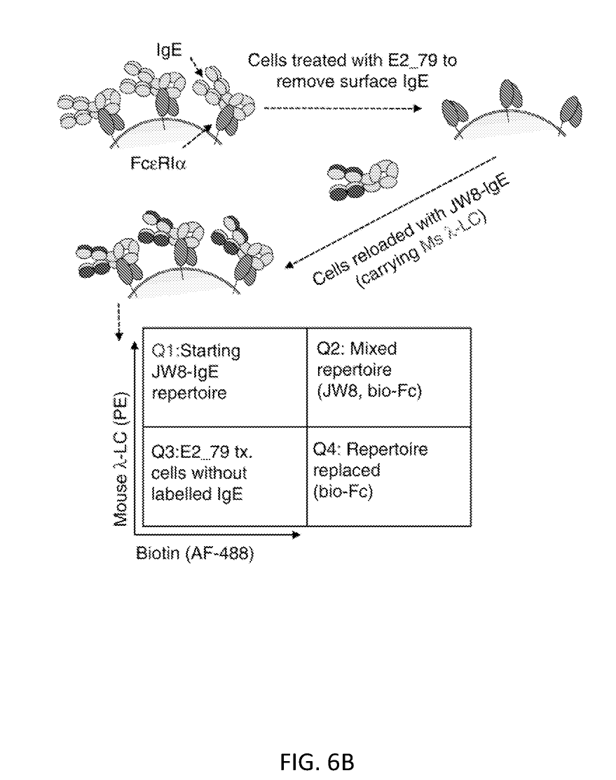

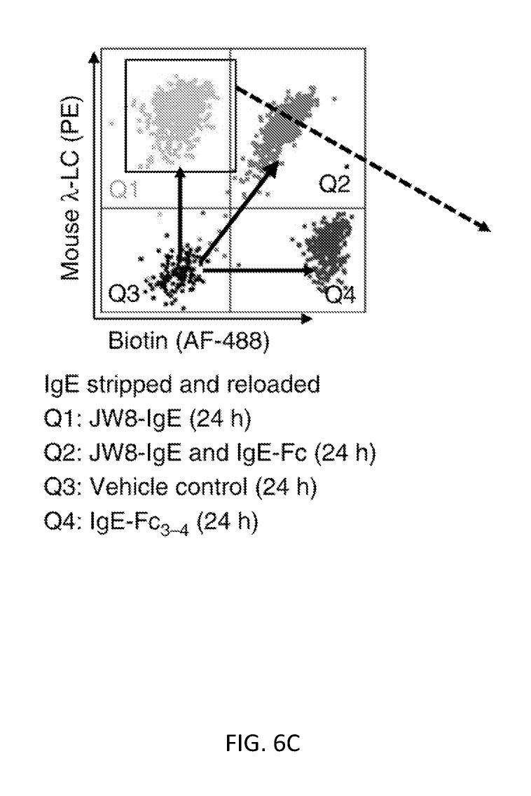

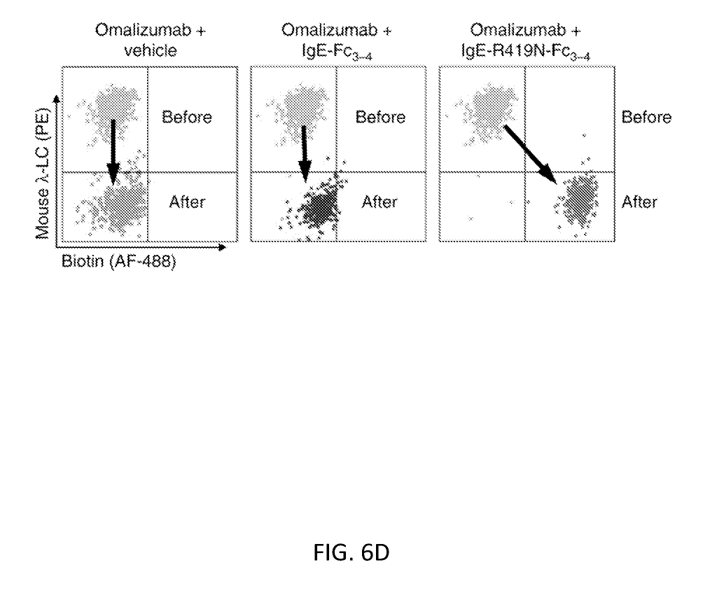

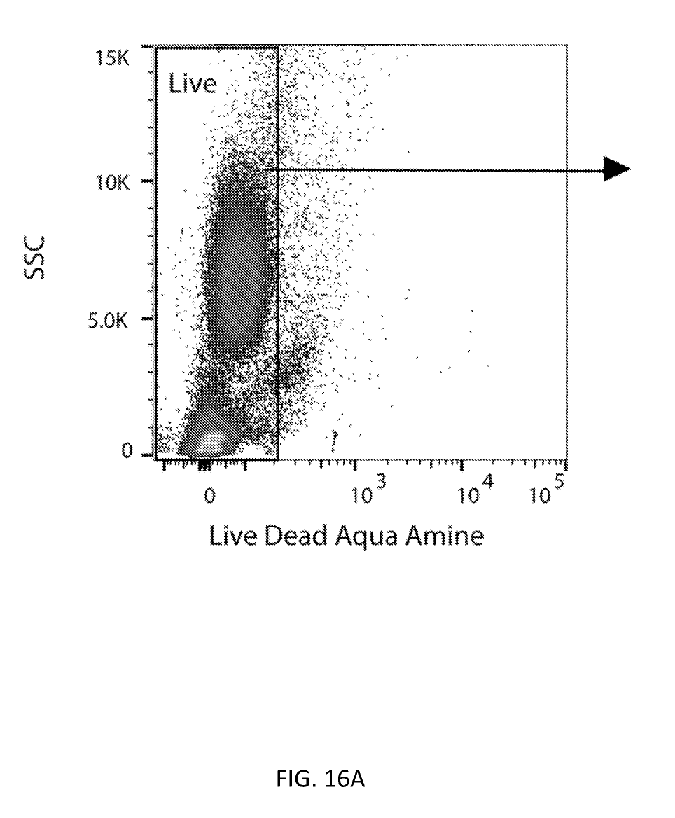

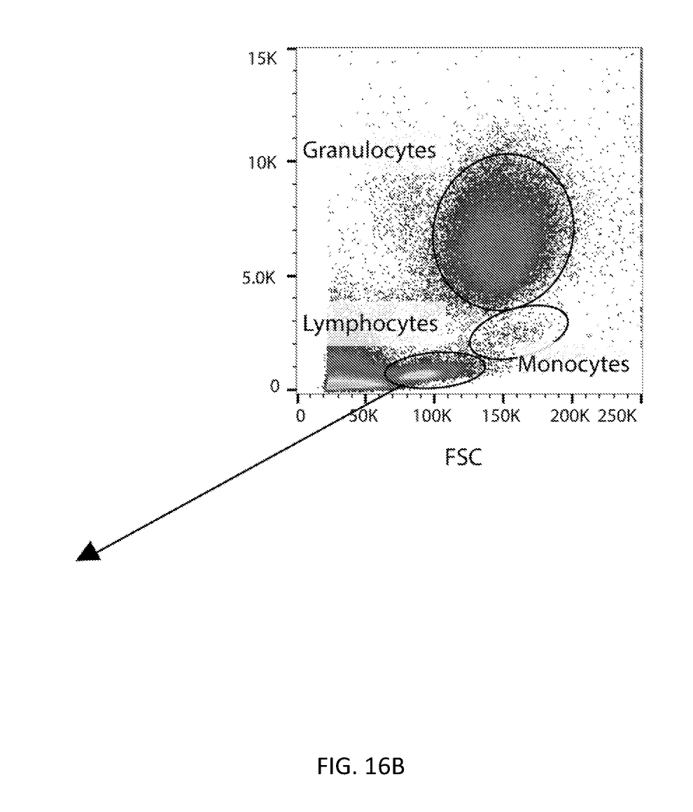

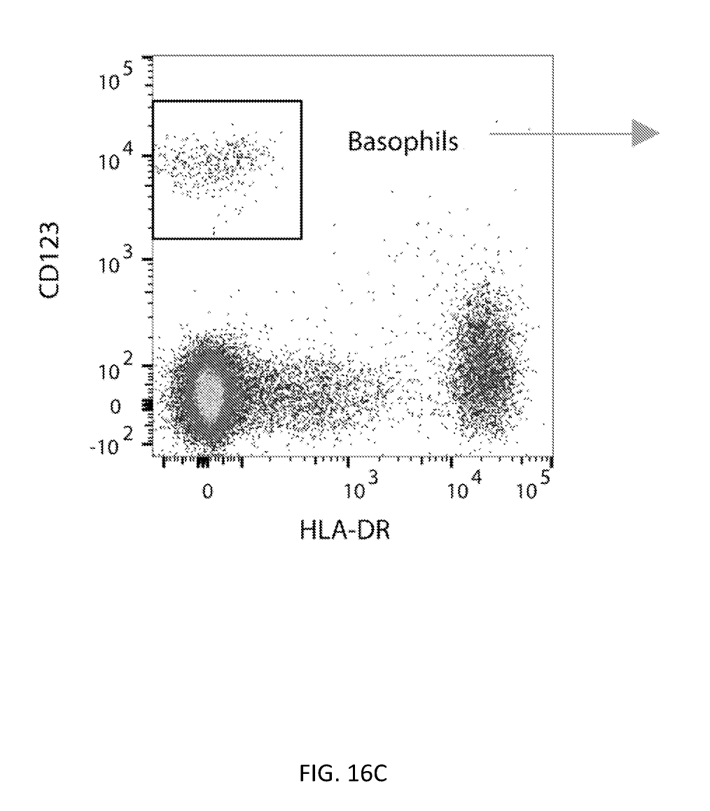

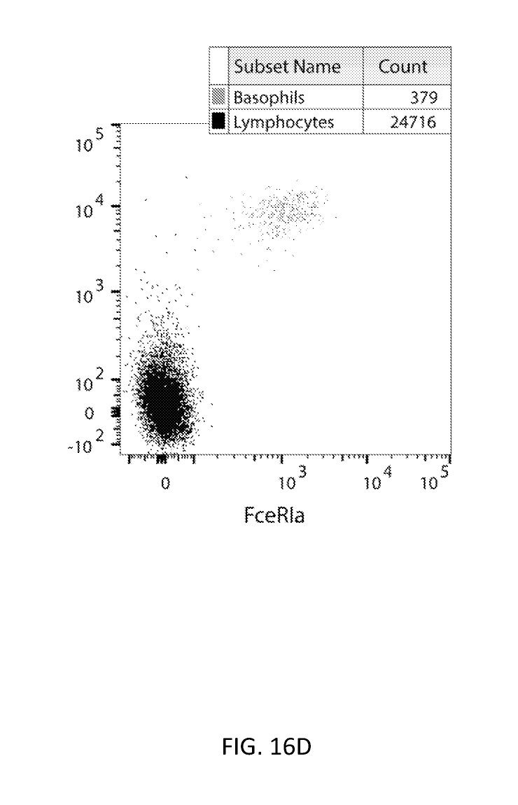

We isolated blood from three donors, and depleted surface IgE on basophils by treating each sample with the disruptive inhibitor E2_79 (FIG. 6A, gating scheme FIG. 16). We then reloaded the cells with JW8-IgE (FIG. 6A), while also verifying that stripped cells could be reloaded with biotinylated IgE-Fc₃₋₄ or IgE-R419N-Fc₃₋₄ species (FIG. 6A). Therefore, we could remove native IgE species, reload cells with homogeneous traceable IgE and subsequently track the removal or exchange of JW8-IgE (FIGS. 6B, 6C). The 1-light-chain-specific staining of JW8 and biotin-specific staining of the IgE-Fc₃₋₄ variants distinguished human basophils that had no JW8-IgE or biotinylated-IgE-Fc₃₋₄, a mix of both species, or a single IgE species (FIGS. 6B, 6C).

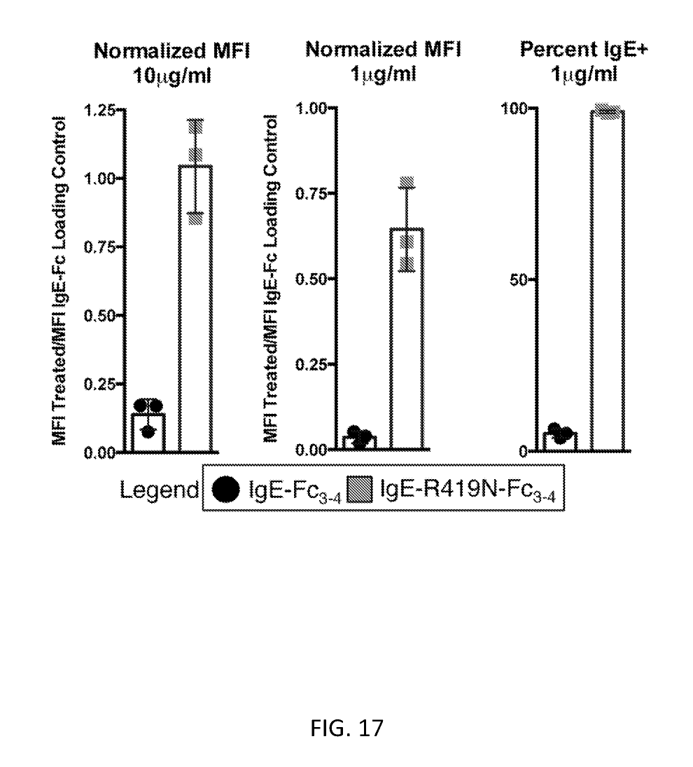

Overnight omalizumab treatment of cells that had been homogenously reloaded with JW8-IgE completely removed the JW8-IgE from the cell surface, as shown by an overlay of the pre- and post-treatment flow cytometry profiles (arrow, FIG. 6D). Co-administration of IgE-Fc₃₋₄ and omalizumab also showed complete depletion of JW8-IgE species but no exchange for IgEFc₃₋₄ at 1 μg ml⁻¹ IgE doses (FIG. 6D). In contrast, co-administration of omalizumab-resistant IgE-R419N-Fc₃₋₄ with omalizumab depleted JW8-IgE surface levels, and facilitated IgE-R419N-Fc₃₋₄ reloading to levels observed in IgE-stripped basophils treated with IgE-R419N-Fc₃₋₄ alone (FIG. 6D). Therefore, co-administration of omalizumab and IgE-R419N-Fc₃₋₄ effectively exchanges the receptor-bound IgE on human basophils for IgE-R419N-Fc₃₋₄ ex vivo. Even when wild-type IgE-Fc₃₋₄ doses far beyond physiologic IgE levels were employed (10 μg ml⁻¹), only minimal reloading of wild-type IgE-Fc₃₋₄ was observed, in contrast to the dramatic reloading of IgE-R419N-Fc₃₋₄ (FIG. 17). This effect was not restricted to FcεRI-expressing basophils. CD23+B-cells treated with omalizumab and IgE-Fc₃₋₄ variants retained surface IgE-R419N-Fc₃₋₄, but were stripped of wild-type IgE-Fc₃₋₄. Furthermore, both wild-type IgE-Fc₃₋₄ and IgE-R419N-Fc₃₋₄ occupancy of the CD23 receptor stabilized surface CD23 and increased CD23 surface levels. Importantly, IgE-mediated stabilization of surface CD23 prevents the release of soluble CD23 (sCD23), a soluble mediator shown to upregulate IgE expression (Cooper et al., supra; Fellmann et al., supra).

IgE-R419N-Fc₃₋₄ and Omalizumab Act Synergistically.

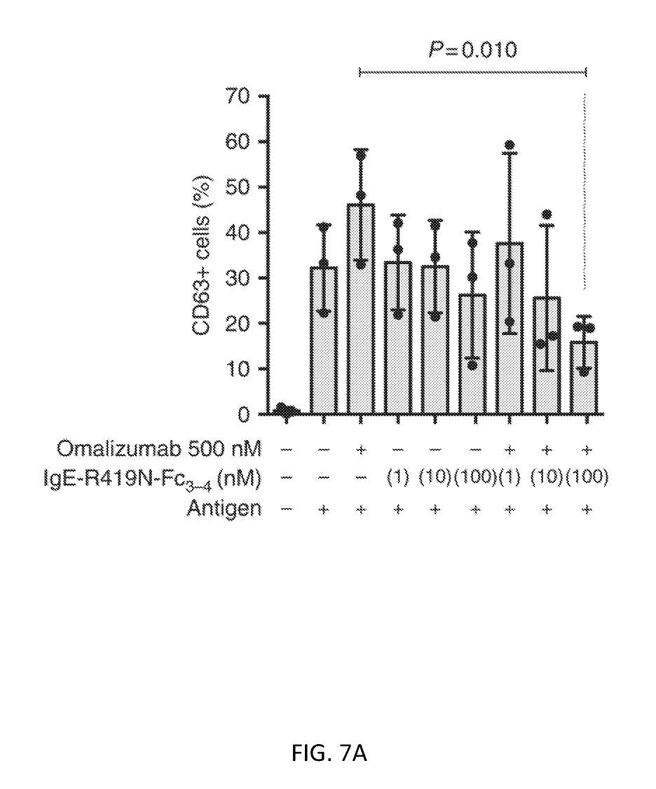

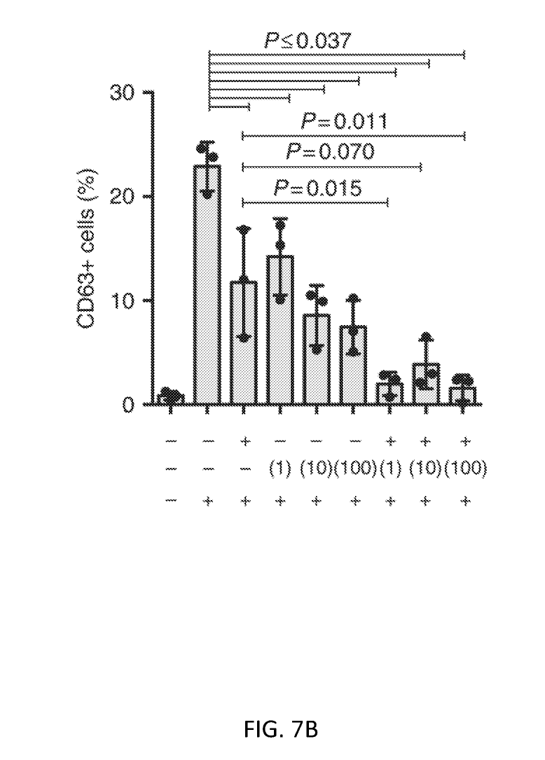

To determine whether IgE-R419N-Fc3-4 could enhance the effect of omalizumab treatment in human cells, we performed basophil-activation tests. Basophils were isolated from healthy donors, treated with DARPᵢₙₛ to remove surface IgE (using the enhanced bi53_79 DARPᵢₙ (Eggel et al., supra)) and reloaded with the 4-hydroxy-3-nitrophenylacetyl (NP)-specific JW8-IgE. The resultant NP-reactive basophils were then left untreated or were treated with omalizumab (at therapeutic concentrations of 500 nM), IgE-R419N-Fc₃₋₄ or combinations of both agents for 3 (FIG. 7A) or 6 days (FIG. 7B) before NP-antigen challenge. Given that IgE:FcεRIα complexes dissociate slowly, and omalizumab is a weak disruptive inhibitor at the therapeutically relevant concentration used, we did not anticipate a rapid response in basophils homogenously reloaded with JW8-IgE. Accordingly, after 3 days, there were no significant reductions in basophil responses as compared with untreated controls (FIG. 7A). Of note, omalizumab transiently increased basophil sensitivity on day 3, although this effect was overcome by day 6, presumably as omalizumab neutralized a greater fraction of JW8-IgE previously bound to cells. Although this effect was not significant, it does fit with clinical studies, which suggest that basophil sensitivity is increased on a per IgE basis during omalizumab therapy (Macglashan et al. (2013) J. Allergy Clin. Immunol. 132:906-911). There was significantly less activation in cells treated with a combination of omalizumab and IgE-R419N-Fc₃₋₄ (100 nM) as compared with omalizumab alone by day 3 (FIG. 7A), suggesting that combination therapy could offset this transient effect in omalizumab-treated samples. By day 6, a significant reduction in basophil activation was observed in all treatment arms as compared with the untreated controls (FIG. 7B). This result was surprising, given that IgE-R419N-Fc₃₋₄ alone was able to inhibit basophil reactivity at 1 nM concentrations (FIG. 7B). Furthermore, in combination with omalizumab, 1 nM IgE-R419N-Fc₃₋₄ almost completely inhibited basophil activation (FIG. 7B). The additive effect of omalizumab (500 nM) and IgE-R419N-Fc₃₋₄ (1 nM) should be minor if each agent acted only as a competitive inhibitor for JW8-IgE:FcεRIα interactions, suggesting that these agents can act synergistically to block basophil activation.

Discussion

Anti-IgE therapy remains an important tool for the management of allergic disorders, and novel second-generation anti-IgE therapies will soon join omalizumab. Yet, the etiology of allergic disorders is complex and optimal therapeutic responses will likely come from treatment regimens that target and modulate multiple immunological processes.

Here we have clarified the structural basis for omalizumab-mediated inhibition of IgE binding to both FcεRI and CD23. This analysis revealed similarities between the disruptive inhibitor E2_79 and omalizumab, and provides a mechanistic framework to develop antibody-based therapies that can accelerate the dissociation of IgE:FcεRIα complexes. Such agents could rapidly disarm basophils and mast cells, allowing them to achieve a therapeutic effect faster.

We have also demonstrated that mutant IgE fragments, in conjunction with omalizumab treatment, can exchange native IgE on human cells for IgE fragments, maintaining the occupancy of both high- and low-affinity IgE receptors. We pursued this approach, given the experimental evidence that basophils increase in sensitivity on a per IgE basis during omalizumab treatment (Zaidi et al. (2010) J. Allergy Clin. Immunol. 125:902-908 e907; MacGlashan et al. (2012) J. Allergy Clin. Immunol. 130:1130-1135; Macglashan et al. (2013) J. Allergy Clin. Immunol. 132:906-911), and that IgE production can be regulated by IgE:CD23 signaling (Cooper et al., supra; Sherr et al., supra; Bonnefoy et al., supra; Aubry et al., supra; Fellmann et al., supra). These observations raise significant questions about the impact of therapeutic IgE depletion, and suggest that homeostatic responses to the loss of IgE could offset or constrain the therapeutic benefit of anti-IgE treatment. Our experimental observations provide a system to test some of these regulatory pathways and could potentially be used as an adjunct therapy with omalizumab. To this end, we have also demonstrated that IgE-R419N-Fc₃₋₄ can act synergistically with omalizumab ex vivo at very low doses to inhibit basophil activation. This finding suggests that simultaneously targeting FcεRI and IgE with competitive inhibitors could enhance therapeutic responses. Prior studies have demonstrated that non-activating ligands can antagonize FcεRI responses to activating ligands by sequestering receptor-proximal signaling components (Torigoe et al. (1998) Science 281:568-572), and it is possible that IgE-R419N-Fc₃₋₄:FcεRI complexes could suppress activation through such mechanisms, in addition to competing for receptor occupancy. Sustained FcεRI receptor occupancy could also suppress homeostatic responses to the loss of IgE on omalizumab-treated basophils.

Finally, the dissociation of allergen-specific IgE on mast cells located in peripheral tissues is slow, and the effectiveness of anti-IgE within these compartments is poorly understood (Beck et al. (2004) J. Allergy Clin. Immunol. 114:527-530). Given that the FcεRI on mast cells has been shown to drive IgE tissue localization (Cheng et al. (2013) Immunity 38:166-175), co-administration of omalizumab and omalizumab-resistant IgE fragments that bind FcεRI may enhance the exchange of allergen-specific IgE in peripheral sites, and contribute to the therapeutic benefit of anti-IgE treatment.

Despite the potential utility of IgE exchange, and promising ex vivo results, the concept is far from clinical application. Unexpected antibody responses to the R419N IgE, although unlikely, could prevent the development of any universally benign IgE variant for exchange therapy, and could induce anaphylactic antibody responses to receptor-bound IgE fragments. Yet, our approach with IgE-R419N-Fc₃₋₄ does have two distinct advantages in this regard. First, N-linked glycosylation events are effective at masking antibody epitopes, and could reduce the immunogenicity of IgE-R419N-Fc₃₋₄ (Wei et al. et al. (2003) Nature 422:307-312). Second, the new antigenic surface generated by truncating IgE to the Cε₃₋₄ domains would be largely inaccessible once bound to FcεRIα, preventing any antibody responses to this epitope from crosslinking receptor-bound IgE fragments. Given that anti-IgE and anti-FcεRIα antibody responses are relatively common, and are not always pathological (Chan et al. (2014) J. Allergy Clin. Immunol. 134:1394-1401), it is possible that such an approach could be well tolerated.

Methods

Preparation of Omalizumab and Recombinant Proteins.

Fab fragments of Omalizumab (Novartis) were prepared by digestion over an immobilized ficin agarose resin (Pierce) in 10-mM citrate buffer with 25 mM cysteine and 5 mM EDTA at pH 6.0 for 5 hours. The Fab fragments were purified in two steps with protein G (Pierce) and gel filtration to yield homogenous omalizumab-Fab. Protein G columns were washed with phosphate buffer over a pH gradient (8.0, 7.0, 6.0 and 5.0) and Fab was eluted with glycine buffer at pH 2.5. Insect-cell-derived IgE⁻Fc3-4, IgE-G335C-Fc₃₋₄ and FcεRIα were expressed in High Five insect cells and purified using Ni-NTA affinity chromatography and further purified using gel filtration on a Superdex 200 10/300 GL column (GE). All insect vectors have been previously published: IgE-Fc₃₋₄ (Wurzburg et al. (2012), supra), IgE-G335C-Fc₃₋₄ (Wurzburg et al. (2012), supra) and FcεRIα (Kim et al. (2012) Anal. Biochem. 431:84-89). Mammalian-derived IgE-Fc₃₋₄ and the R419N mutant, used in all cell-based assays, were cloned into the pYD7 vector (National Research Council (NRC), Canada) with a vascular endothelial growth factor (VEGF) signal sequence from the pTTVH8G vector (NRC). Constructs were transfected using 25-kDa linear polyethylenimine (Polysciences), and transiently expressed in suspension HEK-6E cells (NRC) for 120 hours according to the supplier's protocols. Cell supernatants were filtered through 0.45-μM Durapore filter (Millipore) and incubated with Ni-NTA resin (Qiagen) for 1 hour at room temperature, washed with 10 resin bed volumes of wash buffer (25 mM imidazole in PBS at pH 7.4) and eluted with 2 resin bed volumes of elution buffer (300 mM imidazole in PBS at pH 7.4). The eluted protein was then concentrated using an Amicon Ultra-15 filter unit (Millipore) and further purified with gel filtration on a Superdex 200 10/300 GL column (GE). Protein used for experiments with human basophils was subsequently buffer-exchanged into sterile PBS pH 7.4 using an Amicon Ultra-15 filter unit (Millipore). The DARPᵢₙₛ E2_79 and bi53_79 were cloned into the pQE-30 expression vector between BamHI and HindIII restriction sites, and expressed in XL-1 blue E. coli (NEB) at 37° overnight following induction with isopropyl-β-D-thiogalactoside. Soluble DARPᵢₙₛ were purified using Ni-NTA affinity chromatography, followed by gel filtration on a Superdex 200 10/300 GL column (GE).

Crystallization Conditions.

Crystals were grown in hanging drops in 0.2 M Lithium Sulfate, 0.1 M Tris pH 8.5 and 41% PEG 400. A volume of 0.1 μl of complex at 9 mg ml⁻¹ was added to 0.1 μl of well solution. Crystals were harvested and frozen in the same crystallization buffer for data collection.

Structure Determination of the IgE:Omalizumab Complex.

Diffraction data were collected in two 3600 sweeps of one crystal on the microfocus beamline 12-2 at the Stanford Synchrotron Radiation Lightsource, and data were indexed, integrated and scaled using the HKL2000 suite (Otwinowski, Z. & Minor, W. in Methods in Enzymology: Macromolecular Crystallography, part A (eds Carter, C. W. JRMS) (Academic Press, 1997)). The omalizumab-C335 crystals grew in the P21 space group with the unit cell dimensions a=100.10 Å, b=107.14 Å, c=151.04 Å and β=95.18°. A molecular replacement solution was obtained using Phaser-MR in the Phenix package version 1.9 (Adams et al. (2010) Acta Crystallogr. D Biol. Crystallogr. 66:213-221) using the models 4GT7 (for C335-IgE-Fc) (Wurzburg et al. (2012), supra) and 4X7S (for omalizumab-Fab) (Jensen et al., supra). Automated model building to improve early models was performed using Phenix AutoBuild, and refinement was performed using rounds of Phenix Refine and manual model building with Coot (version 0.8.1, Emsley et al. (2004) Acta Crystallogr. D Biol. Crystallogr. 60:2126-2132). NCS restraints were identified automatically in phenix. refine by sequence similarity and default root mean squared deviation tolerance of <2 Å. NCS restraints were applied in early rounds of refinement and removed in final rounds of refinement. The final model was refined to 2.5 Å (Table 1) and was validated using MolProbity and Phenix comprehensive validation. The model had Ramachandran-favored conformations in >97% of residues and 0.2% of residues were outliers. The relatively high B-factors after refinement are expected, given the high-average Wilson B-factor (61.89).

Calculation Volume Overlaps.

Structural alignments with omalizumab:IgE and E2_79:IgE complexes were made with the Cε3 domain at site 2 of the FcεRIα complex. The alignment with the CD23:IgE complex was made with either Cε3 domain within the CD23 complex (similar results were found with both alignments in this symmetric complex). The coordinates of each IgE-binding molecule (FcεRIα, omalizumab, E2_79 and CD23) from these aligned complexes were then loaded into pdbset (CCP4) and centered within an arbitrarily defined unit cell to accommodate the full chain. The coordinates of the aligned and transformed structures were then input into sfall (CCP4) to generate an atom map from the polypeptide chain (solvent atoms and ligands were not included), and input into mapmsk (CCP4) to make a map mask. The resulting map masks were input into overlapmap (CCP4) using the MAP INCLUDE function, keeping only density of overlapping regions of each inhibitor with the receptor complex in question. The volume of the resulting overlap maps were then calculated with the volumes tool in Chimera (Pettersen et al., supra).

Biotinylation of Proteins.

IgE-Fc₃₋₄ or R419N-IgE-Fc₃₋₄ was biotinylated using EZ-Link Sulfo-NHS-LC-biotin (Pierce), with a 30-fold molar excess of sulfo-NHS-LC-biotin for 30 minutes at room temperature. The reaction was stopped using 1.0 M Tris pH 7.4, and proteins were dialyzed overnight into PBS pH 7.4, and sterile-filtered with 0.22-μm filter.

Basophil IgE Exchange Experiments.

Blood was drawn from two healthy volunteers and a third volunteer with a history of food allergy ranging in age from 21 to 37 (the protocol for this study was approved by the Institutional Review Board of Stanford University, and all informed consent was obtained from all subjects). Blood was collected in heparin vacutainer tubes (BD), and washed in 10 blood volumes of BF buffer (RPMI-1640 (Life Technologies) supplemented with 10% fetal calf serum (FCS; Gibco) and 1% penicillin/streptomycin (Gibco)). Washed blood cells were then suspended in their original blood volume in BF buffer and treated with or without E2_79 at 25 μM for 2 hours at 37° C. to remove surface IgE. Stripped cells were then reloaded with IgE-JW8 at 120-300 ng ml⁻¹ as estimated from supplier's supplied concentration range (AbD Serotec) or were left untreated. These cells were then treated overnight at 37°, as specified in the figure legends with omalizumab, IgE-Fc variants or vehicle controls. The following day cells were washed three times with BF buffer at 4° C. and stained for analysis.

Flow Cytometry for Basophil IgE Exchange.

After washing, treated cells and controls were incubated with human Fc-block (BD), and stained with the following antibodies as described in figure legends (at 1:100 dilution unless stated): IgE-FITC (eBiosciences clone: IgE21), FcεRIα-APC (eBiosciences clone AER-37 at 1:50 dilution), anti-biotin AF-488 (eBiosciences clone: BK-1/39), anti-mouse-lambda-light-chain PE (BioLegend clone: RML-42), CD123 PE-Cy5 (BD clone: 9F5 at 1:20 dilution), HLA-DR PE-Cy7 (BioLegend clone L243 at 1:80 dilution), CD203c BV421 (BioLegend clone: NP4D6 at 1:50 dilution), CD19-PE (BD clone: HIB19 at 1:50), CD23-BV421 (BioLegend EBVCS-5 at 1:50 dilution) and Aqua Live Dead stain (Life Technologies). Stained cells were then lysed with RBS lysis buffer (BioLegend) for 5 min at room temperature, and washed with FACS buffer (PBS pH 7.4 supplemented with 10% FCS). Data were collected on a DxP FACSCAN from Cytek Development in Fremont, Calif. (10 colors with three lasers—488, 639, 407) using FlowJo CE and analyzed using FlowJo (version 10).

SPR Assays.

SPR measurements were conducted on a BIAcore X100 device and evaluated with the BIAevaluation software (GE Healthcare, Fairfield, USA). For kinetic analysis of different IgE fragments on omalizumab and FcεRIα, 1,000 response units of omalizumab or rhFcεRIα were immobilized in acetate buffer (pH 4.5 for omalizumab and pH 4.0 for rhFcεRIα) on flow cell 2 of a CM5 chip (GE Healthcare). Flow cell 1 was activated and deactivated without immobilization, according to the manufacturer's protocol. The different IgE fragments (produced as described above) or full-length Sus11 IgE were diluted in HBS-EP+running buffer (GE Healthcare) and injected for 120 seconds at a constant flow rate of 10 μl min⁻¹. Dissociation was assessed for 240 seconds under constant buffer flow.

For each measurement, the chip surface was regenerated with 50 mM NaOH. Individual sensorgram curves were exported to Excel, and graphs were prepared with the GraphPad Prism 5.0 software (GraphPad Software, La Jolla, USA). In all experiments, unspecific binding to flow cell 1 was subtracted from the signal on flow cell (Okada et al. (2010) Clin. Exp. Immunol. 160:1-9).

Functional Assay with Primary Human Basophils.

Human primary basophils were isolated from whole blood of volunteers with approval from the local ethics committee (KEK Bern). Informed consent was obtained from all donors in accordance with the Helsinki Declaration. Human basophils were isolated from three different donors, with total IgE levels ranging from 35 to 78 kU l⁻¹ by using Percoll density centrifugation of dextran-sedimented supernatants with further purification with the Miltenyi basophil isolation kit II (Miltenyi Biotec, Bergisch Gladbach, Germany), as previously described (Tschopp et al. (2006) Blood 108:2290-2299). Total IgE levels of the donors were determined using ImmunoCAP (Phadia, Uppsala, Sweden). Purified primary human basophils were seeded at 0.5×10⁵ cells per well in a 96-well plate in 50 μl of RPMI containing 10% heat-inactivated FCS, 100 IU ml⁻¹ penicillin and 100 μg ml⁻¹ streptomycin (medium). Cells were kept in a cell incubator at 37° C., 5% CO₂. For desensitization, cells were treated with 50 μM disruptive anti-IgE inhibitor bi53_79 for 8 hours in the presence of 10 ng ml⁻ rhIL-3 and subsequently washed three times with 150 μl PBS to remove dissociated IgE and anti-IgE inhibitor from the supernatant. For resensitization, cells were incubated with 100 nM JW8-IgE (NBS-C BioScience, Vienna, Austria) for 2 hours in the presence of 10 ng ml⁻¹ rhIL-3. Subsequently, cells were washed two times with 150 μl PBS and treated with omalizumab, IgE-R419N-Fc₃₋₄ or a combination of these molecules for 3 or 6 days at the indicated concentrations. For determination of basophil activation, cells were stimulated with 1-1,000 ng ml⁻¹ NIP(7)BSA (BioSearch Technologies, Petaluma, USA) in the presence of 10 ng ml⁻¹ rhIL-3 for 30 minutes at 37° C., 5% CO₂. Subsequently, cells were stained with 10 μl anti-CD63 FITC anti-CCR3-PE staining mix (FK-CCR Flow CAST Buhlmann Laboratories AG, Schonenbuch, Switzerland) for 20 minutes at room temperature. At least 3×10³ basophils were acquired on a FACSCalibur device. Data were analyzed with the FlowJo V10 software (TreeStar, Ashland, Oreg.).

Although preferred embodiments of the subject invention have been described in some detail, it is understood that obvious variations can be made without departing from the spirit and the scope of the invention as described herein.

TABLE 1
Data collection and refinement statistics.

| | IgE-G335C-Fc₃₋₄:omalizumab-Fab | |
|---|---|---|
| Data collection | | |
| Space group | P 1 21 1 | |
| Cell dimensions | | |
| a, b, c (Å) | 100.10, 107.14, 151.04 | |
| α, β, γ (°) | 90.00, 95.18, 90.00 | |
| Resolution (Å) | 37.61-2.50 | (2.59-2.50) |
| R_{merge} | 0.115 | (2.009) |
| CC1/2 | 0.999 | (0.756) |
| CC* | 1.000 | (0.928) |
| I/σI | 22.09 | (1.94) |
| Wilson B-factor | 61.89 | |
| Completeness (%) | 97.80 | (95.73) |
| Redundancy | 26.9 | (23.0) |
| Refinement | | |
| Resolution (Å) | 2.50 | |
| No. of reflections (work/test) | 107,541/1,512 | |
| R_{work}/R_{free} | 22.07/23.9 | |
| No. of atoms | | |
| Macromolecule | 20,083 | |
| Ligand/ion | 484 | |
| Water | 59 | |
| B-factors | | |
| Macromolecule | 70.40 | |
| Ligand/ion | 98.20 | |
| Water | 61.10 | |
| r.m.s.d. | | |

| | |
|---|---|
| bond lengths | 0.003 |
| bond angles | 0.812 |

r.m.s., root mean square; r.m.s.d., root mean square deviation.

1. An omalizumab-resistant immunoglobulin E (IgE) variant comprising a heavy chain polypeptide with a substitution of an amino acid corresponding to Arg-92, numbered relative to the reference sequence of SEQ ID NO: 1, wherein the substitution interferes with binding of the IgE variant to omalizumab.

2. The omalizumab-resistant IgE variant of claim 1, wherein the amino acid corresponding to Arg-92, numbered relative to the reference sequence of SEQ ID NO: 1, is replaced with an Asn.

3. The omalizumab-resistant IgE variant of claim 1, wherein the substitution introduces a glycosylation site into the IgE variant.

4. The omalizumab-resistant IgE variant of claim 3, wherein the amino acid corresponding to Arg-92, numbered relative to the reference sequence of SEQ ID NO: 1, is glycosylated.

5. The omalizumab-resistant IgE variant of claim 1 comprising an amino acid sequence having at least 70% sequence identity to the amino acid sequence of SEQ ID NO: 2.

6. The omalizumab-resistant IgE variant of claim 5 comprising an amino acid sequence having at least 80% sequence identity to the amino acid sequence of SEQ ID NO: 2.

7. The omalizumab-resistant IgE variant of claim 6 comprising an amino acid sequence having at least 90% sequence identity to the amino acid sequence of SEQ ID NO: 2.

8. The omalizumab-resistant IgE variant of claim 7 comprising an amino acid sequence having at least 95% sequence identity to the amino acid sequence of SEQ ID NO: 2.

9. The omalizumab-resistant IgE variant of claim 8 comprising the amino acid sequence of SEQ ID NO: 2.

10. The omalizumab-resistant IgE variant of claim 9, wherein Asn-92 is glycosylated.

11. A composition comprising the omalizumab-resistant immunoglobulin E (IgE) variant of claim 1 and a pharmaceutically acceptable excipient.

12. The composition of claim 11, further comprising one or more additional agents selected from the group consisting of an antihistamine, an antileukotriene, a corticosteroid, a bronchodilator, and an anti-IgE therapeutic agent.

13. The composition of claim 12, wherein the anti-IgE therapeutic agent is omalizumab.

14. A method of performing anti-IgE therapy comprising administering to a subject a therapeutically effective amount of omalizumab in combination with a therapeutically effective amount of an omalizumab-resistant IgE variant.

15. The method of claim 14, wherein the subject has an IgE-mediated disorder.

16. The method of claim 14, wherein the omalizumab-resistant IgE variant is administered according to a daily dosing regimen.

17. The method of claim 14, wherein the omalizumab-resistant IgE variant is administered intermittently.

18. The method of claim 14, wherein the omalizumab-resistant IgE variant is administered for a period of time before administration of the omalizumab.

19. The method of claim 18, wherein the omalizumab-resistant IgE variant is administered for one week before the first dose of omalizumab is administered to the subject.

20. The method of claim 14, wherein the omalizumab-resistant IgE variant is administered for a period of time after administration of the omalizumab.

21. The method of claim 20, wherein the omalizumab-resistant IgE variant is administered for one week after the last dose of omalizumab is administered to the subject.

22. The method of claim 14, wherein the omalizumab is administered once every 2 to 4 weeks.

23. The method of claim 14, wherein the omalizumab-resistant IgE variant is administered subcutaneously.

24. The method of claim 14, wherein the subject is human.

25. The method of claim 14, wherein the omalizumab-resistant IgE variant comprises an amino acid sequence having at least 70% sequence identity to the amino acid sequence of SEQ ID NO: 2.

26. The method of claim 25, wherein the omalizumab-resistant IgE variant comprises an amino acid sequence having at least 80% sequence identity to the amino acid sequence of SEQ ID NO: 2.

27. The method of claim 26, wherein the omalizumab-resistant IgE variant comprises an amino acid sequence having at least 90% sequence identity to the amino acid sequence of SEQ ID NO: 2.

28. The method of claim 27, wherein the omalizumab-resistant IgE variant comprises an amino acid sequence having at least 95% sequence identity to the amino acid sequence of SEQ ID NO: 2.

29. The method of claim 28, wherein the omalizumab-resistant IgE variant comprises the amino acid sequence of SEQ ID NO: 2.

30. The method of claim 14, wherein the omalizumab-resistant IgE variant binds to an Fc receptor on the surface of a mast cell or basophil in the subject.

31. The method of claim 14, wherein the omalizumab-resistant IgE variant exchanges with or replaces a harmful allergy-inducing IgE on a basophil in the subject.

32. The method of claim 14, wherein the omalizumab-resistant IgE variant exchanges with or replaces a harmful allergy-inducing IgE on an IgE+, HLA-DR+, or FcεRIα lymphocyte in the subject.

33. The method of claim 14, further comprising treating the subject with one or more other drugs or agents for treating a IgE-mediated disorder selected from the group consisting of an antihistamine, a antileukotriene, a corticosteroid, a bronchodilator, and an anti-IgE therapeutic agent.

34. A kit comprising the composition of claim 11 and instructions for treating an IgE-mediated disorder.

35. The kit of claim 34, further comprising means for delivering said composition to a subject.

36-37. (canceled)
